(19) United States
(12) Patent Application Publication
(10) Pub. No.: US 2024/0245718 A1
(43) Pub. Date: Jul. 25, 2024

(54) MEDIUM MOLECULAR WEIGHT HEPARIN FOR USE IN THE TREATMENT OF ENDOTHELIOPATHY

(71) Applicant: GLYCOS BIOMEDICAL LTD, London (GB)

(72) Inventor: Pervinder Singh Bhogal, London (GB)

(73) Assignee: GLYCOS BIOMEDICAL LTD, London (GB)

(21) Appl. No.: 18/290,533

(22) PCT Filed: May 16, 2022
(86) PCT No.: PCT/EP2022/063222

§ 371 Date: Nov. 14, 2023

(30) Foreign Application Priority Data
May 14, 2021 (GB) 2106972.9

(51) Int. Cl. A61K 31/727; A61P 7/02

(52) U.S. Cl. CPC A61K 31/727; A61P 7/02

(57) ABSTRACT

Medium molecular weight heparin for use in the treatment of disease, specifically, in the treatment of endotheliopathy in a patient having a plasma von Willebrand factor antigen to ADAMTS13 ratio of at least about 2. Also, a pharmaceutical formulation and a kit comprising a medium molecular weight heparin, and a method of treating endotheliopathy, comprising administering to a patient in need of treatment a therapeutically effective amount of medium molecular weight heparin, wherein the patient has a plasma von Willebrand factor antigen to ADAMTS13 ratio of at least about 2.

### FIELD OF THE INVENTION

The present invention relates to the treatment of endotheliopathy. Particularly, medium molecular weight heparins for use in the treatment of endotheliopathy.

### BACKGROUND OF THE INVENTION

The vertebrate vasculature system consists of arteries, veins and capillaries. Blood flow through the vasculature is dynamic allowing for the maintenance of homeostasis through the delivery of essential elements such as oxygen and leukocytes to the tissues most in need. Blood flow is controlled through the dilation and constriction of the blood vessels. The endothelial cells which line the inside of the lumen of the vasculature act as a monolayer to form the endothelium; the endothelium sits upon a layer of smooth muscle cells. These smooth muscle cells either constrict or relax which results in constriction of the blood vessels (vasoconstriction) or dilation of the blood vessels (vasodilation), respectively. In the case of haemorrhage, blood flow can be controlled through thrombus formation. To reach sites of inflammation, leukocytes must pass from the blood through the endothelial cells to reach inflamed tissue. The regulation of vasoconstriction, vasodilation, vascular permeability, and thrombus formation are therefore crucial for the regulation of homeostasis.

The endothelial cells lining the blood vessel are more than simple constituents of the vessel wall. Endothelial cells produce and release vasoactive substances that relax and constrict blood vessels. For example, endothelial cells produce nitric oxide (NO) in response to sheer stress or stimuli, such as acetylcholine, histamine and thrombin. The NO then diffuses to the smooth muscle cells surrounding the endothelium to initiate vasodilation. Reactive oxygen species, which are released in response to inflammatory stimuli, can increase endothelial permeability and promote leukocyte adhesion to the endothelial cells via the expression of adhesion molecules. This serves to drive the influx of leukocytes to sites of inflammation. Moreover, the endothelium provides a surface for thrombus formation. Thus, it is the endothelial cells that play a crucial role in the regulation of homeostasis.

Endothelial dysfunction, or endotheliopathy, can therefore have dire consequences as blood flow, oxygen delivery, the immune response and, therefore, homeostasis are impaired. Endotheliopathy is characterised by decreased NO bioavailability. This can result in the increased expression of adhesion molecules on the endothelial surface, thereby initiating leukocyte recruitment to the vascular wall. Thus, inflammation of the endothelium, or endothelialitis, is observed in endotheliopathy. This can result in a defective lining of the blood vessels by the endothelium, resulting in the exposure of the subendothelial matrix to clotting factors in the blood. Consequently, platelet aggregation and thrombus formation occur, resulting in a potentially lethal blood clot.

Endotheliopathy may be caused by a number of diseases and is typically viewed as a symptom rather than a cause of disease. Consequently, treatments have focussed on targeting the causative disease rather than the endotheliopathy itself. As a result of this, treatments for endotheliopathy are lacking. However, it is now suggested that an underlying endotheliopathy may actually drive disease severity and morbidity and endotheliopathy plays a much larger role than previously thought. Therefore, treating the endotheliopathy and not just the causative disease may increase patient survival.

Herein, endotheliopathy and endothelialitis are used interchangeably. While endotheliopathy may be caused by a large number of diseases and/or conditions as described herein, endotheliopathy with reference to COVID-19 or SARS-CoV-2 will primarily be discussed herein. The skilled person will understand that this discussion is merely to provide an example and should not be considered limiting on the present invention.

In late 2019, in Wuhan, China a new beta-coronavirus (Severe Acute Respiratory Syndrome Coronavirus 2, SARS-CoV-2) was identified, causing coronavirus disease 2019 (COVID-19). Thereafter, a rapid geographical progression of COVID-19 culminated in the WHO declaring a pandemic in March 2020 (1). The clinical manifestations of those infected with SARS-CoV-2 range from the asymptomatic patient to a more severe pneumonia, which can lead to acute respiratory distress syndrome (ARDS) and multi-organ failure. The majority of symptomatic patients will experience a mild-to-moderate form of the disease, which most commonly does not require hospitalisation (2-4). However, there is a cohort of patients that can progress to the more severe form of the disease, where evolution of symptoms/clinical manifestations can take up to 2 weeks, starting from an initial prodromal stage through to ARDS (3). It is now known that the subgroup of patients that become critical and require ventilation or extracorporeal membrane oxygenation (ECMO) have very poor outcomes with high rates of mortality that approach 90% (5).

Since the disease was first described it has affected more than 90 million individuals globally. The pathophysiological pathways remain unclear; accordingly, management is supportive. Disease-modifying therapeutics which could be instigated whilst awaiting a specific anti-viral drug or vaccination are lacking. Several lines of evidence point towards endothelial dysfunction as a key pathophysiological mechanism in COVID-19. Prior to the current pandemic, markers of endothelial dysfunction have been shown to be correlated with disease severity and mortality in patients with sepsis (6-9). Recently Varga et al. (10) demonstrated a widespread endothelialitis that affected pulmonary, renal, gastrointestinal and hepatic vessels on post mortem examination of three patients with COVID-19. In one of the cases the authors reported ‘most of the small vessels appeared congested’ and in another case the patient died from bowel ischaemia with evidence of underlying endothelialitis.

Recently, two proposed haemostatic mechanisms have provided insight into an improved understanding of ARDS based on a molecular pathogenesis associated with endotheliopathy that promotes inflammation and coagulation disorder in sepsis and other critical illnesses (11-14): one is the “two-activation theory of the endothelium” in which an endothelial pathogenesis activates the inflammatory pathway and microthrombotic pathway, whilst the other is a novel “two-path unifying theory” of haemostasis in which haemostasis initiates thrombogenesis and promotes micro-thrombogenesis, leading to vascular microthrombotic disease (VMTD) (11,13,15). These two theories are in congruity with one another since the endothelium contributes to initial haemostasis and triggers the molecular mechanisms of thrombogenesis. ARDS is often associated with sepsis from a variety of different causes and has been seen in severe acute respiratory syndrome (SARS) due to SARS-CoV (16), Middle East respiratory syndrome (MERS) due to MERS-CoV (17) and now COVID-19. Sepsis-associated ARDS often develops with other organ dysfunction such as encephalopathy (18), hepatic failure (19)(20), acute renal failure, and acute necrotizing pancreatitis (21). This multi-organ involvement suggests ARDS may not be the primary disease but is part of an on-going systemic pathogenic mechanism triggered by an infection or another critical illness.

On this basis the underlying physiologic alteration of multi-organ failure in sepsis and other critical illnesses is identified as circulatory dysfunction occurring as a result of an endotheliopathy associated VMTD (EA-VMTD) (14,15). Therefore, the infection triggers an insult to the endothelium, causing an endotheliopathy. This then results in disseminated microthrombosis (DIMT), which may trigger, for example, local hypoxaemia, systemic hypoxia, and/or ischaemia, and as mentioned earlier COVID-19 is now known to be associated with an endothelialitis (10).

A case series of COVID-19 pulmonary autopsies revealed that, alongside diffuse alveolar damage, numerous localised platelet-rich micro-thrombi and foci of haemorrhage were present in the lungs (22). The authors posited a pulmonary-localised thrombotic microangiopathy as key to the pathogenesis of COVID-19 with others also suggesting the micro-thrombosis is a critical driver in the disease process (23). These microcirculatory changes have been clearly demonstrated in the lungs, kidneys and the liver using contrast enhanced ultrasound (24,25). Similar findings have also been seen in the brain (26). Therefore, there is a growing body of evidence that COVID-19 appears to cause an endothelialitis and a diffuse and widespread microthrombosis.

Hypercoagulability and COVID-19 is now widely accepted and studies have shown abnormal levels of D-Dimers with higher levels associated with more severe disease and increased odds ratio of in-hospital mortality (27-30). Several case reports have noted acute pulmonary emboli in patients with COVID-19 pneumonia in the absence of major predisposing factors for venous thromboembolism formation (27,31,32). More recently Panigada et al. have shown that, in addition to raised D-Dimer levels, there was a marked increase in the levels of Factor VIII and von Willebrand factor (VWF) (33). An increase of over 500% in VWF and >350% increase in Factor VIII levels were reported by Escher et al (34) in relation to COVID-19. Furthermore, it has been demonstrated that patients with a thrombocytopaenia were at over 5-fold increased risk of severe disease and those with the lowest platelet counts were associated with the highest mortality (33,35,36) Therefore, both hypercoagulability and thrombocytopaenia appear to be harbingers of severe disease and mortality.

Von Willebrand Factor (VWF) is a multimeric plasma glycoprotein that plays a critical role in haemostasis and thrombosis mediating platelet adhesion to injured and activated vessels. It is synthesized only in megakaryocytes and endothelial cells (ECs) and it is interesting to note that SARS-CoV can directly infect both of these cell types (22,36).

The vast majority of VWF found in the plasma is derived from the VWF synthesised within the ECs, where it is stored within the Weibel Palade Bodies (WPB). Although restricted to ECs there are differences in the synthesis of VWF within the different vascular beds of the body, with the small vessels of the lung and brain expressing higher levels of VWF than similar sized vessels of the liver or kidney and higher levels in venous rather than arterial ECs (37). A major portion of the VWF stored in the WPBs of endothelial cells is made up of ultra-large VWF (ULVWF). These ultra-large VWF multimers are more adhesive than the smaller VWF multimers in the circulation (38). Upon secretion, ULVWF can spontaneously bind platelets. Inflammatory cytokines such as Interleukin-1 and tumour necrosis factor (TNF)-alpha can trigger the exocytosis of WPBs with release of their contents. Thus, plasma level of VWF can be used as a marker of endothelial activation and vascular inflammation and raised levels of VWF have been shown to associate with ARDS and sepsis, and to correlate independently to mortality (39,40).

Upon secretion from ECs, the secreted VWF, which partly enters the circulation and partly binds to the endothelium, is sensitive to shear stress. This shear stress unfolds the VWF and exposes sites for platelet binding, self-association as well as for cleavage via the enzyme ADAMTS13. It has previously been shown these VWF molecules can self-associate into long ‘strings’ in the direction of flow, both arterial and venous, that bind to platelets and are adherent to the endothelium (41-43). A protease, ADAMTS13, cleaves VWF and ULVWF, perfusion of which over these platelet-VWF strings led to them being rapidly removed from the circulation (41). The ULVWF multimers released from the WPBs have a lower shear stress for unfolding and therefore may represent the initiating molecules for this self-assembly process which leads to hyper-adhesive strings capturing platelets. The binding of platelets to the VWF occurs via the GP Ib receptor. The binding site for this receptor is usually not exposed when the VWF is in its globular form and therefore cannot bind to platelets. Once VWF unfurls, secondary to shear stress, the binding site is exposed and binds with high affinity to platelets. The binding of platelets to VWF may cause a conformational change leading to activation of the integrin GPIIbIIIa (also known as α2bβ3) and promoting platelet-platelet as well as platelet-VWF cross binding. For this reason, the use of standard anti-platelet agents is likely to be ineffective (Aspirin or P2Y12 inhibitors) or only partially effective in mitigating this pathological process as was suggested by the cohort study of Tremblay et al (44).

This ability to form VWF-platelet rich thrombi in the microvasculature is the hallmark of acquired thrombotic thrombocytopaenic purpura (TTP) in which auto-antibodies to ADAMTS13 are present. It has also been shown that Interleukin-6 (IL-6) can inhibit the cleavage of ULVWF—platelet strings (45). Furthermore, the synthesis of ADAMTS13, at least in cultured cells, is dramatically inhibited by a variety of cytokines including IL-6 and TNF-alpha (46). This suggests that the cytokine storm, and particularly IL-6, may propagate the microthrombosis. However, this also suggests that if intervention is implemented early and there is no spike in the release of cytokines the disease may be more manageable and the rapid deterioration in the clinical status of patients can be averted.

There is now a significant body of evidence to suggest that there is very marked imbalance in the VWF:ADAMTS13 ratio as well as in the levels of high molecular weight VWF multimers (equivalent to ULVWF) in COVID-19. As mentioned earlier very high levels of VWF have been shown previously with the earliest case report to mention this surge in the levels of VWF being that of Escher et al (34). Subsequently, Goshua et al. (47) demonstrated that reported marked elevations in plasma VWF concentrations in patients admitted with COVID-19 with increased levels associated with disease severity—mean VWF antigen levels of 565±199% vs 278±133 for those admitted to an intensive care unit (ICU) compared to those not admitted to ICU (p<0.0001). Next, Rauch et al. (48) looked at the progression of patients with COVID-19 in relationship to their admission VWF. Those with the highest VWF levels required greater levels of oxygen support whereas those patients that had normal VWF levels did not require admission to hospital nor supplementary oxygen (n=10).

Shortly after the Rauch et al. publication Ladikou et al. (49) showed an increase in the VWF antigen levels of patients with COVID-19 admitted to the ICU with a positive correlation seen in the VWF levels and the age of the patients. They reported a median VWF Antigen level of 350% however, and crucially, they also showed a markedly reduced level of ADAMTS13 (49.7%), suggesting loss of the VWF cleaving protease that ordinarily degrades large VWF multimers and reduces its activity. They speculated that excess release of VWF seen in COVID-19 patients leads to depletion of ADAMTS13 and contributes to the prothrombotic state. Further analysis of their data showed that median VWF levels were significantly higher in patients that died (477%) compared to the ones that remained alive (335%) (p=0.015).

Helms et al. (50) recently published a multicentre prospective cohort study in France, assessing thrombotic risk in COVID-19 patients, which showed that VWF and factor VIII were considerably increased. In conjunction with this data showing increases in VWF and reductions in ADAMTS13 there is further research to show that the VWF:ADAMTS13 ratio is substantially deranged. Huisman et al. (51) were the first to show a mean VWF antigen:ADAMTS13 ratio of 8.5 (normal 0.5-2) from 12 patients admitted to the ICU. Subsequently, Mancini et al (52) demonstrated similar findings with an elevated von Willebrand Factor antigen (VWF:Ag) to ADAMTS13 activity ratio that was strongly associated with disease severity with the worst ratio, 8.3, seen in those patients that required high intensity care (intubation and mechanical ventilation) compared to those requiring low intensity care, 3.42 (p<0.001).

Most recently, Philippe et al. (53) published their results from a cohort of 208 patients admitted to two centres in Paris of whom 23 had only mild symptoms and were treated as outpatients. They found that only VWF:Ag scaled according to clinical severity, with levels significantly higher in critical patients (median 507%, IQR 428-596) compared to non-critical patients (288%, 230-350, p<0.0001) or COVID-19 outpatients (144%, 133-198, p=0.007). In a univariable analysis model a VWF:Ag level over 423% at admission was significantly associated with higher in-hospital mortality (OR 89.7 95% CI 25.9-567.4, p<0.001) which remained very significant in a multivariable analysis model adjusted on age, BMI, D-Dimer and C-reactive protein (CRP) (odds ratio, OR 25.6, 95% CI 5.6-198.2, p<0.001). More importantly they showed that VWF high molecular weight multimers (HMWM) were significantly higher in critical patients (median ratio 1.18, IQR 0.86-1.09) compared to non-critical patients (0.96, 1.04-1.39, p<0.001). Furthermore, the levels of HMWM (ratio) (OR 116, 95% CI 10.2-1943, p<0.001) was one of the most significantly associated with in-hospital mortality.

It is possible to develop a unifying theory that is triggered by an endotheliopathy and endothelialitis, which causes the release of VWF and ULVWF resulting in the formation of microthrombi. This then leads to hypoxia and the process can be accentuated by the ‘cytokine storm’ and release of IL-6, which inhibits and reduces the functions of ADAMTS13, resulting in a cascade of disseminated microthrombosis and multi-organ dysfunction and failure. It has also been suggested that this microvascular thrombosis at the pulmonary level is the origin of right ventricular dysfunction (54). This mechanism can account for many of the findings currently being observed including the high D-Dimer levels (high because of the huge levels of microthrombosis), high levels of Factor VIII and VWF (released from the WPBs in response to an endothelial insult), the microthrombosis and atypical ARDS picture being seen (55), as well as the widespread clinical picture of pulmonary, neurological and gastrointestinal symptoms. The endothelialitis and microthrombosis we suggest may also explain why patients with a pre-existing endotheliopathy and micro-arteriopathy (e.g., secondary to diabetes mellitus, hypertension, or obesity) are at increased risk of severe COVID-19 (29,56). Similarly, there is a rapidly growing body of evidence linking patients with low levels of ADAMTS13 and high levels of VWF with a variety of diseases that pre-dispose to a poor outcome after infection with SARS-CoV-2 and to its variable presentation (57-64). The use of standard anti-platelet medication (aspirin or P2Y12 inhibitors) is also likely to be ineffective given that the interaction between VWF and platelets activates the GP2b3a receptor. Although the inhibition of VWF-platelet binding via the GP1b receptor, using either caplacizumab or anfibatide, would be an attractive option and has been suggested (65) these drugs are not in widespread use and clinical experience with them is extremely limited. Similarly, they carry a significant haemorrhage profile.

Thus, there is a need for a treatment of endotheliopathy per se.

### SUMMARY OF THE INVENTION

Heparin is a naturally occurring, highly sulphated polysaccharide characterised by a wide molecular weight range of polysaccharide chains. Heparin acts at a variety of different ligands with varied actions. Heparin is a member of the glycosaminoglycan carbohydrate family and consists of repeating disaccharide units of GlcAβ1-4GlcNAcα1-4 with poly-disperse sulfation, N-acetylation and uronosyl epimerization. Heparin is highly heterogenous. Heparin isolated from natural sources contains polysaccharide chains with molecular weights ranging from about 3000 Da to about 30,000 Da. This is known as unfractionated heparin (UFH). UFH can be enzymatically or chemically treated to deliver shorter polysaccharide chains. The products of the chemically or enzymatically treated UFH can be affinity purified to yield fractionated heparin where the molecular weight of the polysaccharides in each fraction can be readily determined. Low molecular weight heparin (LMWH) contains polysaccharide chains in the range of about 4000 Da to about 8000 Da.

In 1991, it was first demonstrated that the intravenous administration of heparin to patients during open heart surgery induced the impairment of VWF-dependent platelet function, without changes in plasma VWF levels (66). This inhibitory effect of heparin on VWF-dependent platelet agglutination was not dependent on the heparin's affinity for anti-thrombin III, but was dependent upon the molecular weight of heparin. From later in vitro experiments, it was found that heparin bound to a specific amino acid sequence within the A1 domain of VWF (residues 569-583), in which basic amino acids are regularly arranged. Heparin binding induced conformational changes in a peptide of this binding site (67). Heparin bound to both activated and inactivated VWF similarly, but did not interfere with VWF binding to collagen. Since the platelet GpIb-binding domain (residues 524-542) is also located in the A1 domain, it was suggested that heparin interferes with VWF binding to platelet GpIb both by steric hindrance and by inducing a conformational change of the domain that results in inhibition of platelets binding.

The structural specificity of the heparin that is responsible for binding to VWF, revolves around key disaccharide units—GlcNS6S-IdoA2S and IdoA2S-GlcNS6S. Further, it has been demonstrated that the assembly of more than 3 units of the disaccharide was crucial for the binding potency. Similarly, although fractionated heparins of lower molecular weight (6100 Da (g/mol)) have shown a higher affinity to binding to the VWF they were less able to inhibit VWF activity compared to UFH. This suggests that a minimum heparin molecular weight and molecular size is important in order to achieve steric hindrance.

These medium molecular weight heparins may have specificity towards inhibiting VWF-GPIb binding, hence stopping microthrombosis, but as they have little effect on anti-thrombin III they have little anti-coagulant effect. Thus, medium molecular weight heparins with a mass of about 11 000 Da (g/mol) represent an ideal treatment option when considering the treatment of patients with pro-thrombotic states that are dependent upon increased VWF levels and endotheliopathies. Furthermore, the results of these earlier studies suggests that low molecular weight heparins are unlikely to work and do not target the GPIb receptor and that UFH, whilst it may contain the sugar moieties that can bind to VWF, is sub-optimal. Moreover, monitoring of UFH is difficult and the other fractions of UFH, e.g. the LMWH fractions, have anticoagulant effects which can result in dangerous bleeding events, which are unpredictable.

Of further interest is the fact that it has recently been shown that SARS-CoV-2 binds to heparin sulphates and in particular requires the IdoA2S-GlcNS6S sugar moiety (74,75). This suggests that exogenous supply of these sugar moieties may inhibit binding to the endogenous heparan sulphates in the lungs and hence act as a potential prophylactic treatment. Taken together, a specialized medium molecular weight heparin (˜11000 Da (g/mol)) with at least 3 units of the GlcNS6S-IdoA2S disaccharide may inhibit viral adherence and replication but also inhibit the microthrombosis triggered by the release of VWF secondary to the endotheliopathy caused by the virus.

As described herein, endotheliopathy may be associated with a number of diseases. The Inventor has found that medium molecular weight heparin can be used to treat endotheliopathy, particularly endotheliopathy in a patient having a high plasma von Willebrand factor level.

Thus, the invention provides medium molecular weight heparin for use in treating endotheliopathy. The medium molecular weight heparin may inhibit von Willebrand factor (VWF). The medium molecular weight heparin may inhibit multimers of VWF, preferably ultra-large VWF. The medium molecular weight heparin may inhibit the binding of platelets to VWF.

Accordingly, in a first aspect, the present invention provides medium molecular weight heparin for use in the treatment of endotheliopathy in a patient. Preferably, wherein the patient has a plasma von Willebrand factor to ADAMTS13 ratio of at least about 2. Alternatively or additionally, the patient may have a von Willebrand factor antigen to ADAMTS13 ratio of at least about 2.

The medium molecular weight heparin may inhibit von Willebrand factor (VWF). The medium molecular weight heparin may inhibit multimers of VWF, preferably ultra-large VWF. The medium molecular weight heparin may inhibit the binding of platelets to VWF.

The patient may have a VWF:ADAMTS13 ratio of at least about 2, of at least about 4, of at least about 8, or of at least about 10. The patient may have a VWF:ADAMTS13 ratio greater than about 2, greater than about 4, or greater than about 8, or greater than about 10. The patient may have a VWF:ADAMTS13 ratio of about 2-16, about 4-12, or preferably about 6-10. A patient having a VWF to ADAMTS13 ratio of greater than about 8 typically indicates severe illness and often is indicative of a patient deteriorating towards death.

The patient may have a VWF antigen:ADAMTS13 ratio of at least about 2, of at least about 4, of at least about 8, or of at least about 10. The patient may have a VWF antigen:ADAMTS13 ratio greater than about 2, greater than about 4, or greater than about 8, or greater than about 10. The patient may have a VWF antigen:ADAMTS13 ratio of about 2-16, about 4-12, or preferably about 6-10. A patient having a VWF antigen to ADAMTS13 ratio of greater than about 8 typically indicates severe illness and often is indicative of a patient deteriorating towards death.

The level of VWF and ADAMTS13 in the patient may be measured using an ELISA. The ratio may be calculated as described by Huisman et al (51). Briefly, the level of the VWF antigen may be determined in international units and the level of ADAMTS13 may be determined in international units and then the ratio of VWF antigen: ADAMTS13 determined.

Normal levels of plasma VWF are in the range of from about 50 IU per dL to about 200 IU per dL. The mean level of plasma VWF in the general population is about 100 IU per dL. High levels of plasma VWF are those of about 200 IU per dL or more, for example from about 200 IU per dL to about 400 IU per dL, from about 225 IU per dL to about 375 IU per dL, from about 250 IU per dL to about 350 IU per dL, from about 275 IU per dL to about 300 IU per dL.

The patient may have a raised VWF antigen level of about 150% or more, of about 175% or more, of about 200% or more, of about 300% or more, of about 350% or more, or about 400% or more, or of about 500% or more. The patient may have a VWF antigen level of up to about 600%, up to about 700%, up to about 800%, or up to about 1000%.

It is noted that levels of plasma VWF may be temporarily raised by infections, inflammation, trauma, and with physical and emotional stressors. Accordingly, the patient may have a non-temporarily raised plasma von Willebrand factor level, for example for at least about six hours, at least about 12 hours, at least about 18 hours or at least about 24 hours. Preferably, the patient may have a raised plasma von Willebrand factor level for at least about one day, at least about two days, at least about three days, at least about four days, at least about five days, at least about six days, or at least about seven days. Even more preferably the patient may have a raised plasma von Willebrand factor level for at least about one week, at least about two weeks, at least about three weeks or at least about four weeks. Yet even more preferably the patient may have a raised plasma von Willebrand factor level for at least about one month, at least about two months, at least about three months, at least about four months, at least about five months, at least about six months or at least about one year. The patient may have a raised plasma von Willebrand factor level for up to about one week, up to about four weeks, up to about two months, up to about four months, up to about six months, or up to about one year.

The medium molecular weight heparin may have a mass of about 11 000 Da (g/mol). The medium molecular weight heparin may comprise at least three units of the GlcNS6S-IdoA2S (or IdoA2S-GlcNS6S) disaccharide.

The endotheliopathy may be caused by COVID-19, viral infection, acute respiratory distress syndrome (ARDS), cancer, bacterial infection, septicaemia, cardiovascular disease, diabetes mellitus, trauma, in particular brain or head trauma, burns, inhalational injury, drugs and drug reactions, haematological conditions, subarachnoid haemorrhage, aneurysmal diseases, stroke, or brain parenchymal haemorrhage. The endotheliopathy may be caused by a viral infection, optionally wherein the viral infection is SARS-CoV-2. The endotheliopathy may be caused by cancer, in particular leukaemia, lymphoma, myeloma, or a solid organ cancer, such as colon cancer, breast cancer, brain cancer, lung cancer, pancreatic cancer, testicular cancer, prostate cancer, cervical cancer, liver cancer, or skin cancer.

The treatment of endotheliopathy by medium molecular weight heparin may inhibit the haematogenous spread of cancer. Human tumour cells can bind to VWF under shear flow conditions with both melanoma and colon cancer cells demonstrating this ability. The immobilized platelets, bound to the VWF, have been shown to mediate tethering, rolling, and the firm adhesion of different cancerous cell lines under flow shear stress. The VWF played a critical role in enabling this firm adhesion of the tumour cells to the immobilized platelets. The existing data suggests that VWF plays an important role in tethering cancerous cells. In addition, the VWF-Platelet binding that occurs as part of the normal thrombosis pathways may further act to allow the coalescence of tumour cells into the VWF-Platelet to form heteroaggregates of VWF+platelets+cancer cells which thereby help in the blood borne (haematogenous) spread of tumour cells. This process may at least in part be caused by the ability of cancer cells to translocate to the vessel wall and thereby spread to other organs once the initial binding to VWF and Platelets has occurred. In addition, various cancers are known to cause an endotheliopathy with the resultant release of UL-VWF. By this mechanism, the tumour triggers the release of UL-VWF that then allows the tethering of platelets and tumour cells and the haematogenous spread of the cancer and the metastatic spread. This cancer induced endotheliopathy also results in an overall increase in the risk of thrombosis in patients with underlying malignancy. Therefore, any treatment aimed at treating an endotheliopathy and inhibiting the binding of platelets and/or tumour cells to VWF would serve a dual purpose of decreasing the risk of malignancy associated thrombosis and also reduce the risk haematogenous metastatic spread.

The medium molecular weight heparin may be administered by an administration method selected from the group consisting of: parenteral, subcutaneous, intravenous, intramuscular, intrathecal, intradermal, intraarterial, intraarticular, cutaneous, transcutaneous, subcutaneous, depot form, for example depot injection, intra-osseus, or inhalation. Preferably, the medium molecular weight heparin administration method is subcutaneous, intravenous or intramuscular. The administration method may be inhalation, optionally via a nebuliser.

The medium molecular weight heparin may be administered at a dose of from about 0.01 mg/kg, from about 0.1 mg/kg, from about 1 mg/kg, from about 5 mg/kg, from about 10 mg/kg, from about 20 mg/kg, from about 30 mg/kg, from about 50 mg/kg, from about 70 mg/kg, from about 80 mg/kg, or from about 100 mg/kg. The medium molecular weight heparin may be administered at a dose of about 500 mg/kg or less, about 300 mg/kg or less, about 200 mg/kg or less, or about 100 mg/kg or less. The medium molecular weight heparin may be administered at a dose of from about 0.01 mg/kg to about 10 mg/kg, preferably from about 0.2 mg/kg to about 10 mg/kg, from about 0.2 mg/kg to about 1.6 mg/kg. The medium molecular weight heparin may be administered as a single dose or as a continuous dose. The medium molecular weight heparin dosage amount may be dependent on the VWF antigen: ADAMTS13 ratio or the overall VWF levels. The skilled person would be able select a suitable amount for a patient based on the VWF antigen: ADAMTS13 ratio or the overall VWF levels.

The medium molecular weight heparin may be comprised in a pharmaceutical formulation. The pharmaceutical formulation may comprise an excipient. The excipient may be selected from the group comprising solvents, co-solvents, buffers, stabilisers, antioxidants, preservatives, chelating agents, emulsifiers, flavourings, lubricants, suspending agents, tonicity adjusting agents, surfactants, solubilising agents, suspending aids, dispersion agents, humectants, thickeners, colouring agent, wetting agent, anti-foaming agent, viscosity modifier, sweeteners and combinations thereof. The pharmaceutical formulation may comprise an additional active agent. The additional active agent may comprise low molecular weight heparin.

The medium molecular weight heparin may comprise a chemical modification. The chemical modification may be selected from the group comprising N-acetylation, N-deacetylation, N-sulfation, O-sulfation, 2-O desulfation, and complete desulfation.

In a second aspect the present invention provides medium molecular weight heparin for use in the treatment of a disease or condition in a patient, wherein the patient has an endotheliopathy characterised by a plasma von Willebrand factor to ADAMTS13 (VWF:ADAMTS13) ratio of at least about 2.

In some embodiments, the present invention provides medium molecular weight heparin for use in the treatment of COVID-19 in a patient, wherein the patient has an endotheliopathy characterised by a plasma VWF:ADAMTS13 ratio of at least about 2.

In some embodiments, the present invention provides medium molecular weight heparin for use in the treatment of viral infection in a patient, wherein the patient has an endotheliopathy characterised by a VWF:ADAMTS13 ratio of at least about 2. The viral infection may be SARS-CoV-2.

In some embodiments, the present invention provides medium molecular weight heparin for use in the treatment of acute respiratory distress syndrome (ARDS) in a patient, wherein the patient has an endotheliopathy characterised by a plasma VWF:ADAMTS13 ratio of at least about 2.

In some embodiments, the present invention provides medium molecular weight heparin for use in the treatment of cancer in a patient, wherein the patient has an endotheliopathy characterised by a VWF:ADAMTS13 ratio of at least about 2. The cancer may be leukaemia, lymphoma, myeloma, or a solid organ cancer, such as colon cancer, breast cancer, brain cancer, lung cancer, pancreatic cancer, testicular cancer, prostate cancer, cervical cancer, liver cancer, or skin cancer.

In some embodiments, the present invention provides medium molecular weight heparin for use in the treatment of bacterial infection in a patient, wherein the patient has an endotheliopathy characterised by a plasma VWF:ADAMTS13 ratio of at least about 2.

In some embodiments, the present invention provides medium molecular weight heparin for use in the treatment of septicaemia in a patient, wherein the patient has an endotheliopathy characterised by a plasma VWF:ADAMTS13 ratio of at least about 2.

In some embodiments, the present invention provides medium molecular weight heparin for use in the treatment of cardiovascular disease in a patient, wherein the patient has an endotheliopathy characterised by a plasma VWF:ADAMTS13 ratio of at least about 2.

In some embodiments, the present invention provides medium molecular weight heparin for use in the treatment of diabetes mellitus in a patient, wherein the patient has an endotheliopathy characterised by a plasma VWF:ADAMTS13 ratio of at least about 2.

In some embodiments, the present invention provides medium molecular weight heparin for use in the treatment of trauma in a patient, wherein the patient has an endotheliopathy characterised by a plasma VWF:ADAMTS13 ratio of at least about 2.

In some embodiments, the present invention provides medium molecular weight heparin for use in the treatment of burns in a patient, wherein the patient has an endotheliopathy characterised by a plasma VWF:ADAMTS13 ratio of at least about 2.

In some embodiments, the present invention provides medium molecular weight heparin for use in the treatment of inhalational injury in a patient, wherein the patient has an endotheliopathy characterised by a plasma VWF:ADAMTS13 ratio of at least about 2.

In some embodiments, the present invention provides medium molecular weight heparin for use in the treatment of drug reactions in a patient, wherein the patient has an endotheliopathy characterised by a plasma VWF:ADAMTS13 ratio of at least about 2.

In some embodiments, the present invention provides medium molecular weight heparin for use in the treatment of haematological conditions in a patient, wherein the patient has an endotheliopathy characterised by a plasma VWF:ADAMTS13 ratio of at least about 2.

In some embodiments, the present invention provides medium molecular weight heparin for use in the treatment of subarachnoid haemorrhage in a patient, wherein the patient has an endotheliopathy characterised by a plasma VWF:ADAMTS13 ratio of at least about 2.

In some embodiments, the present invention provides medium molecular weight heparin for use in the treatment of aneurysmal diseases in a patient, wherein the patient has an endotheliopathy characterised by a plasma VWF:ADAMTS13 ratio of at least about 2.

In a third aspect the present invention provides medium molecular weight heparin for use in the treatment of a disease or condition in a patient, wherein the patient has an endotheliopathy characterised by a plasma von Willebrand factor antigen to ADAMTS13 (VWF antigen:ADAMTS13) ratio of at least about 2.

In some embodiments, the present invention provides medium molecular weight heparin for use in the treatment of COVID-19 in a patient, wherein the patient has an endotheliopathy characterised by a plasma VWF antigen:ADAMTS13 ratio of at least about 2.

In some embodiments, the present invention provides medium molecular weight heparin for use in the treatment of viral infection in a patient, wherein the patient has an endotheliopathy characterised by a VWF antigen:ADAMTS13 ratio of at least about 2. The viral infection may be SARS-CoV-2.

In some embodiments, the present invention provides medium molecular weight heparin for use in the treatment of acute respiratory distress syndrome (ARDS) in a patient, wherein the patient has an endotheliopathy characterised by a plasma VWF antigen:ADAMTS13 ratio of at least about 2.

In some embodiments, the present invention provides medium molecular weight heparin for use in the treatment of cancer in a patient, wherein the patient has an endotheliopathy characterised by a VWF antigen:ADAMTS13 ratio of at least about 2. The cancer may be leukaemia, lymphoma, myeloma, or a solid organ cancer, such as colon cancer, breast cancer, brain cancer, lung cancer, pancreatic cancer, testicular cancer, prostate cancer, cervical cancer, liver cancer, or skin cancer.

In some embodiments, the present invention provides medium molecular weight heparin for use in the treatment of bacterial infection in a patient, wherein the patient has an endotheliopathy characterised by a plasma VWF antigen:ADAMTS13 ratio of at least about 2.

In some embodiments, the present invention provides medium molecular weight heparin for use in the treatment of septicaemia in a patient, wherein the patient has an endotheliopathy characterised by a plasma VWF antigen:ADAMTS13 ratio of at least about 2.

In some embodiments, the present invention provides medium molecular weight heparin for use in the treatment of cardiovascular disease in a patient, wherein the patient has an endotheliopathy characterised by a plasma VWF antigen:ADAMTS13 ratio of at least about 2.

In some embodiments, the present invention provides medium molecular weight heparin for use in the treatment of diabetes mellitus in a patient, wherein the patient has an endotheliopathy characterised by a plasma VWF antigen:ADAMTS13 ratio of at least about 2.

In some embodiments, the present invention provides medium molecular weight heparin for use in the treatment of trauma in a patient, wherein the patient has an endotheliopathy characterised by a plasma VWF antigen:ADAMTS13 ratio of at least about 2.

In some embodiments, the present invention provides medium molecular weight heparin for use in the treatment of burns in a patient, wherein the patient has an endotheliopathy characterised by a plasma VWF antigen:ADAMTS13 ratio of at least about 2.

In some embodiments, the present invention provides medium molecular weight heparin for use in the treatment of inhalational injury in a patient, wherein the patient has an endotheliopathy characterised by a plasma VWF antigen:ADAMTS13 ratio of at least about 2.

In some embodiments, the present invention provides medium molecular weight heparin for use in the treatment of drug reactions in a patient, wherein the patient has an endotheliopathy characterised by a plasma VWF antigen:ADAMTS13 ratio of at least about 2.

In some embodiments, the present invention provides medium molecular weight heparin for use in the treatment of haematological conditions in a patient, wherein the patient has an endotheliopathy characterised by a plasma VWF antigen:ADAMTS13 ratio of at least about 2.

In some embodiments, the present invention provides medium molecular weight heparin for use in the treatment of subarachnoid haemorrhage in a patient, wherein the patient has an endotheliopathy characterised by a plasma VWF antigen:ADAMTS13 ratio of at least about 2.

In some embodiments, the present invention provides medium molecular weight heparin for use in the treatment of aneurysmal diseases in a patient, wherein the patient has an endotheliopathy characterised by a plasma VWF antigen:ADAMTS13 ratio of at least about 2.

For the avoidance of doubt, embodiments described herein related to the medium molecular weight heparin of the first aspect of the invention also apply mutatis mutandis to the medium molecular weight heparin of the second and third aspects of the invention.

Medium molecular weight heparins for use in the treatment of endotheliopathy as defined in the first aspect of the invention are particularly advantageous as said heparins can inhibit the microthrombosis triggered by the release of VWF secondary to the endotheliopathy caused by any disease or condition. When the cause of the endotheliopathy is SARS-CoV-2, said heparins can additionally inhibit viral adherence and replication.

In a fourth aspect, the invention provides a kit comprising medium molecular weight heparin for use in the treatment of endotheliopathy, in particular according to the first or second or third aspects of the invention.

In a fifth aspect, the invention provides a method of treating endotheliopathy, the method comprising administering to a subject in need of treatment a therapeutically effective amount of medium molecular weight heparin, wherein the patient has plasma VWF:ADAMTS13 ratio of at least about 2.

In a sixth aspect, the invention provides the use of the medium molecular weight heparin as defined in the first or second or third aspect for the manufacture of a medicament for the treatment of endotheliopathy.

For the avoidance of doubt, embodiments described herein related to the medium molecular weight heparin of the first aspect of the invention also apply mutatis mutandis to the second to sixth aspects.

### BRIEF DESCRIPTION OF THE FIGURES

The invention will now be described with reference to the following figures which are intended to be non-limiting.

FIG. 1 Graph of activity of Low Molecular Weight (LMW) heparin, Unfractionated (UF) heparin and Medium Molecular Weight (MMW) heparin against Factor IIa.

FIG. 2 Graph of activity of LMW heparin, UF heparin and MMW heparin against Factor X.

FIG. 3 Light Transmission Aggregometry (LTA) trace of inhibition of VWF-induced platelet aggregation with 5 μM, 10 μM and 15 μM doses of MMW heparin against a vehicle control.

FIG. 4 Light Transmission Aggregometry (LTA) trace of inhibition of VWF-induced platelet aggregation with a 15 μM dose of MMW heparin.

FIGS. 5A and 5B Effect of MMWH and LMWH on VWF-dependent platelet agglutination.

FIG. 6 LTA trace of inhibition of VWF-induced platelet agglutination with 5 μM, 10 μM and 20 μM MMWH.

FIG. 7 LTA trace of inhibition of VWF-induced platelet agglutination with 5 μM and 10 μM MMWH.

FIG. 8 LTA trace of inhibition of VWF-induced platelet agglutination with 10 μM MMWH and anti-VWF mAb.

FIG. 9 LTA trace of inhibition of VWF-induced platelet agglutination with 5 μM and 20 μM MMWH and anti-VWF mAb.

FIG. 10 LTA trace of inhibition of VWF-induced platelet agglutination with 10 μM LMWH and anti-VWF mAb.

FIG. 11 LTA trace of inhibition of VWF-induced platelet agglutination with 20 μM LMWH.

FIG. 12 Statistical analysis of MMWH, LMWH and mAb inhibition of VWF-induced platelet agglutination (area under curve and slope)

FIG. 13 LTA trace of inhibition of VWF-induced platelet agglutination with 5 μM, 10 μM and 20 μM MMWH with non-ristocetin agonists: A. ADP as agonist; B. Collagen as agonist; C. TRAP6 as agonist.

FIG. 14 Graphs of MMWH (0 μM, 5 μM, 10 μM and 20 μM) inhibition of VWF-induced platelet aggregation in the presence of agonists: A. ADP; B. Collagen; C. TRAP-6.

### DETAILED DESCRIPTION OF THE INVENTION

Throughout this specification, one or more aspects of the invention may be combined with one or more features described in the specification to define distinct embodiments of the invention.

In the discussion that follows, reference is made to a number of terms, which are to be understood to have the meanings provided below, unless a context expressly indicates to the contrary.

References herein to a singular of a noun encompass the plural of the noun, and vice-versa, unless the context implies otherwise.

Throughout this specification the word “comprise”, or variations such as “comprises” or “comprising”, will be understood to imply the inclusion of a stated element or integer, or group of elements or integers, but not the exclusion of any other element or integer, or group of elements or integers. The term “comprising” includes within its ambit the term “consisting” or “consisting essentially of”.

The term “consisting” or variants thereof is to be understood to imply the inclusion of a stated element or integer, or group of elements or integers, and the exclusion of any other element or integer or group of elements or integers.

The term “consisting essentially of” or variants thereof is to be understood to imply the inclusion of a stated element, integer or step, or group of elements, integers or steps, and that further components may be present, but only those not materially affecting the essential characteristics of the formulation, composition, or compound.

The term “about” herein, when qualifying a number or value, is used to refer to values that lie within ±5% of the value specified.

The terms “treatment” and “therapy” define the therapeutic treatment of a patient, in order to reduce or halt the rate of progression of a disorder or condition, or to ameliorate or cure the disorder or condition. Prophylaxis of a disorder or condition as a result of treatment or therapy is also included.

As used herein, the term “patient” preferably refers to a mammal. Typically, the mammal is a human.

von Willebrand factor (VWF) is a blood glycoprotein involved in haemostasis. VWF is a large multimeric glycoprotein present in blood plasma and produced constitutively as ultra-large VWF in endothelium (in the Weibel-Palade bodies), megakaryocytes (α-granules of platelets), and subendothelial connective tissue. The basic VWF monomer is a 2050-amino acid protein.

A disaccharide is a sugar whose molecules contain two monosaccharide residues.

A low molecular weight heparin is defined herein as a heparin with an average molecular weight of from about 4000 Da (g/mol) to about 8000 Da (g/mol). A medium molecular weight heparin is defined herein as a heparin with an average molecular weight of from greater than about 8000 Da (g/mol) to about 13000 Da (g/mol).

The endotheliopathy may be caused by any disease. In particular, the endotheliopathy may be caused by COVID-19, viral infection, acute respiratory distress syndrome (ARDS), cancer, bacterial infection, septicaemia, cardiovascular disease, diabetes mellitus, trauma, in particular head trauma, burns, inhalational injury, drugs and drug reactions, haematological conditions, subarachnoid haemorrhage, aneurysmal diseases, stroke or brain parenchymal haemorrhage.

The infection may be bacterial, fungal, or parasitic. The infection may be bacterial. The bacterial infection may be Actinomyces israelii, Bacillus anthracis, Bacteroides fragilis, Bordetella pertussis, Borrelia burgdoferi, Borrelia garinii, Borrelia afzelaii, Borrelia recurrentis, Brucella abortus, Brucella canis, Brucella melitensis, Brucella suis, Campylobacter jejuni, Chlamydia pneumoniae, Chlamydia trachomatis, Chlamydophilia psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Klebsiella pneumoniae, Legionella pneumophila, Leptospira species, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Nocardia asteroides, Rickettsia rickettsii, Salmonella, Shigella, Spirochaetes Staphylococcus, Streptococcus, Treponema pallidum, Vibrio cholerae, or Yersinia pestis.

The infection may be fungal. The fungal infection may be Aspergillus, Blastomyces, Candida, Coccidioides, Cryptococcus neoformans, Cryptococcus gattii, Histoplasma, mucormycetes, Tinea cruris, Tinea corporis, or Tinea pedis.

The infection may be parasitic. The parasitic infection may be protozoan eye infection, Chagas' disease, leishmaniasis, toxoplasmosis, giardiasis, malaria, microsporidiosis, or Rhinosporidiosis. Preferably, the parasitic infection is malaria.

The viral infection may be SARS-CoV-2. SARS-CoV-2 is the virus responsible for the disease COVID-19. COVID-19 can result in ARDS. The endotheliopathy may be caused by SARS-CoV-2 infection. The endotheliopathy may be caused by COVID-19. The endotheliopathy may be caused by ARDS.

The endotheliopathy may be caused by cancer. The cancer may be leukaemia, lymphoma or myeloma. Alternatively, or additionally the cancer may be solid organ cancer, for example colon cancer, breast cancer, brain cancer, lung cancer, pancreatic cancer, testicular cancer, prostate cancer, cervical cancer, liver cancer, or skin cancer.

The endotheliopathy may be cause by haematological conditions, for example Thrombotic thrombocytopenic purpura, anaemia, or sickle cell disease.

Dysfunctional endothelial cells may allow for the passage of tumour cells circulating in the blood to pass into the tissues. Thus, treating the endotheliopathy may prevent the haematogenous spread of blood borne cancers. The treatment of the endotheliopathy may inhibit the haematogenous spread of cancer. The medium molecular weight heparin may inhibit the haematogenous spread of cancer.

Biomarkers of endotheliopathy may include raised von Willebrand factor (VWF) levels, ultra-large von Willebrand factor (ULVWF) levels, Factor VIII levels as well as other markers such as Syndecan 1, VWF antigen, VWF activity, VWF multimers, ADAMTS13 levels, platelet counts, VCAM-1, ICAM-1, P-selectin levels, VWF:ADAMTS13 ratio or VWF antigen:ADAMTS13 ratio. Preferably, a biomarker of endotheliopathy is the ratio of VWF:ADAMTS13 or VWF antigen: ADAMTS13.

The patient may have raised plasma von Willebrand factor (VWF) levels compared to a healthy control subject. The patient may have sustained high levels of plasma VWF compared to a healthy control. The levels of plasma VWF may be raised compared to a healthy control subject over a period of at least about one day, at least about two days, at least about three days, at least about four days, at least about five days, at least about six days, or preferably at least about one week. The levels of plasma VWF may be raised compared to a healthy control subject for a period of up to about one week, up to about four weeks, up to about two months, up to about four months, up to about six months, or up to about one year.

For example, the level of plasma VWF may be raised to at least about 50 nmol/L, preferably at least about 60 nmol/L, even more preferably at least about 70 nmol/L or yet even more preferably at least about 90 nmol/L. The level of plasma VWF may be raised to about 130 nmol/L, to about 150 nmol/L, or to about 200 nmol/L. The level of plasma VWF may be raised to at least about 50 nmol/L for at least about one day, at least about two days, at least about three days, at least about four days, at least about five days, at least about six days, at least about one week, at least about one month or at least about one year. The level of plasma VWF may be raised to at least about 60 nmol/L for at least about one day, at least about two days, at least about three, days, at least about four days, at least about five days, at least about six days, at least about one week, at least about one month or at least about one year. The level of plasma VWF may be raised to at least about 70 nmol/L for at least about one day, at least about two days, at least about three days, at least about four days, at least about five days, at least about six days, at least about one week, at least about one month or at least about one year. The level of plasma VWF may be raised to at least about 90 nmol/L for at least about one day, at least about two days, at least about three days, at least about four days, at least about five days, at least about six days, at least about one week, at least about one month or at least about one year. The plasma VWF level may be measured using an Enzyme-Linked Immunosorbent Assay (ELISA).

Alternatively or additionally, the patient may have a plasma von Willebrand factor level of about 200 IU pr dL or more for at least about six hours, at least about 12 hours, at least about 18 hours or at least about 24 hours. Preferably, the patient may have a plasma von Willebrand factor level of about 200 IU pr dL or more for at least about one day, at least about two days, at least about three days, at least about four days, at least about five days, at least about six days, or at least about seven days. Even more preferably the patient may have a plasma von Willebrand factor level of about 200 IU pr dL or more for at least about one week, at least about two weeks, at least about three weeks or at least about four weeks. Yet even more preferably the patient may have a plasma von Willebrand factor level of about 200 IU pr dL or more for at least about one month, at least about two months, at least about three months, at least about four months, at least about five months, at least about six months or at least about one year.

Vascular endothelial function can be assessed in the coronary and peripheral circulations. Non-invasive tests for the assessment of coronary endothelial function include Doppler echocardiography where blood flow is measured in response to pharmacological or physiological stimuli. Other tests include positron emission tomography and phase-contrast magnetic resonance imaging. However, the gold standard test involves invasive quantitative coronary angiography to examine changes in diameter in response to intracoronary infusions of endothelium-dependent vasodilators such as acetylcholine. Assessment of the endothelium in the peripheral circulation includes brachial artery ultrasound and strain-gauge venous impedance plethysmography.

Binding of medium molecular weight heparin to VWF may be assessed by a competitive binding assay. Heparin-Sepharose beads may be incubated with labelled VWF, for example ¹²¹I-vWF, for a period of time to allow the labelled VWF to bind to the immobilized heparin. Varying concentrations of the medium molecular weight heparin may then be added and the amount of displaced labelled VWF determined. Other methods to determine medium molecular weight heparin binding to VWF may include surface plasmon resonance, biolayer interferometry, isothermal titration calorimetry, fluorescence polarisation binding assays, ELISA and microscale thermophoresis.

The inhibition of platelets binding to VWF may be assessed by ristocetin-induced agglutination of fixed platelets. Platelets may be incubated with the medium molecular weight heparin and citrate treated plasma (a VWF source). Ristocetin may then be added, and platelet agglutination then determined. The MMWH may fully inhibit VWF-induced platelet aggregation at a concentration of 15 μM when measured by a ristocetin-induced platelet aggregation assay. Other methods to determine the inhibition of VWF binding to platelets may include ELISA, fluorescence assisted cell sorting, dynamic light scattering, or flow chamber assays.

The medium molecular weight heparin may have a mass in the range of greater than about 8000 Da (g/mol) to about 13 000 Da (g/mol), preferably about 10 000 Da (g/mol) to about 12 000 Da (g/mol). The medium molecular weight heparin may have a mass of about 11 000 Da (g/mol). The medium molecular weight heparin may comprise polysaccharide chains with an average molecular mass in the range of about 9000 Da (g/mol) to about 13 000 Da (g/mol), preferably about 10 000 Da (g/mol) to about 12 000 Da (g/mol). The medium molecular weight heparin may comprise polysaccharide chains with an average molecular mass of about 11 000 Da (g/mol). The molecular weight of the medium molecular weight heparin may be determined by mass spectrometry or size exclusion chromatography, for example.

The medium molecular weight heparin may be chemically synthesised. The medium molecular weight heparin may be enzymatically synthesised. High pressure liquid chromatography may be used to purify the medium molecular weight heparin.

The medium molecular weight heparin may comprise at least three units of a GlcNS6S-IdoA2S disaccharide, for example at least four units, at least five units, at least six units, at least eight units, or at least ten units. The medium molecular weight heparin may comprise less than or equal to 25 units of the GlcNS6S-IdoA2S disaccharide, for example less than or equal to 20 units. The presence of the units of the GlcNS6S-IdoA2S disaccharide may be determined by an antibody, mass spectrometry, or inferred from chemical and enzymatical studies. The GlcNS6S-IdoA2S units may be ordered in succession.

The medium molecular weight heparin may comprise at least three units of a IdoA2S-GlcNS6S disaccharide, for example at least four units, at least five units, at least six units, at least eight units, or at least ten units. The medium molecular weight heparin may comprise less than or equal to 25 units of the IdoA2S-GlcNS6S disaccharide, for example less than or equal to 20 units. The presence of the units of the IdoA2S-GlcNS6S disaccharide may be determined by an antibody, mass spectrometry, or inferred from chemical and enzymatical studies. The IdoA2S-GlcNS6S units may be ordered in succession. The number of IdoA2S-GlcNS6S units may be tailored to provide a desired anti-VWF activity and/or standard anticoagulant activity.

The medium molecular weight heparin may comprise UA2S-GlcNS6S, UA2S-GlcNS, UA-GlcNAc. The medium molecular weight heparin may comprise at least about 60% UA2S-GlcNS6S, UA2S-GlcNS, and UA-GlcNAc. The medium molecular weight heparin may comprise at least about 45%, preferably at least about 48%, preferably at least about 49%, preferably at least about 60% UA2S-GlcNS6S. The medium molecular weight heparin may comprise up to about 60%, preferably up to about 70%, preferably up to about 85% UA2S-GlcNS6S. The medium molecular weight heparin may comprise at least about 4%, preferably at least about 5%, preferably at least about 6%, preferably at least about 10% UA2S-GlcNS. The medium molecular weight heparin may comprise up to about 15%, preferably up to about 20% UA2S-GlcNS. The medium molecular weight heparin may comprise at least 4%, preferably at least 5%, preferably at least 6%, preferably at least about 10% UA-GlcNAc. The medium molecular weight heparin may comprise up to about 15%, preferably up to about 20% UA-GlcNAc. In some embodiments the medium molecular weight heparin may comprise at least 49.2% UA2S-GlcNS6S, 5.4% UA2S-GlcN and 5.4% UA-GlcNAc. In some embodiments the medium molecular weight heparin may comprise at least 82% UA2S-GlcNS6S, 9% UA2S-GlcNS and 9% UA-GlcNAc. The percentage composition of UA-GlcNAc comprising the medium molecular weight heparin may be enriched compared to unfractionated heparin.

The medium molecular weight heparin may be administered by an administration method selected from parenteral, subcutaneous, intravenous, intramuscular, intrathecal, intradermal, intraarterial, or intraarticular, cutaneous, transcutaneous, subcutaneous, depot form, for example depot injection, intra-osseus, or inhalation. Preferred methods of administration comprise subcutaneous, intravenous, intramuscular or inhalation.

Previous studies have looked at UFH as a nebulised agent in a variety of conditions. Small human studies indicate that nebulised UFH limits pulmonary fibrin deposition, attenuates progression of acute lung injury and hastens recovery (69). Early-phase trials in patients with acute lung injury and related conditions found that nebulised UFH reduced pulmonary dead space, coagulation activation, microvascular thrombosis, improved lung injury and increased time free of ventilatory support (70-73). In a pre-pandemic double-blind randomised study in 256 critically ill ventilated patients, nebulised UFH limited progression of lung injury including acute respiratory distress syndrome and accelerated return to home in survivors. Thus, the medium molecular weight heparin may be administered by inhalation via a nebuliser.

Heparin dosage is typically measured in “Howell Units”. One unit of heparin (the “Howell unit”) is an amount approximately equivalent to 0.002 mg of pure heparin, which is the quantity required to keep 1 ml of cat's blood fluid for 24 hours at 0° C. The medium molecular weight heparin may be administered at a bolus does of about 5000 units, followed by about 1200 to about 1600 units per hour optionally delivered by an infusion pump. The medium molecular weight heparin may be administered at a dose of about 18 units/kg to about 5000 units/kg. Preferably, the medium molecular weight heparin may be administered at a dose of about 100 units/kg to about 800 units/kg. Alternatively, the medium molecular weight heparin may be administered at a dose of about 18 units/kg to about 75 units/kg. The medium molecular weight heparin may be administered at a dose of about 5000 units, about 4000 units, about 3000 units, about 2000 units, about 1000 units or about 500 units every 12 hours. The medium molecular weight heparin may be administered at a dose of about 5000 units every 12 hours.

The medium molecular weight heparin may be administered at a dose of about 3 units to about 5000 units, for example from about 6 units to about 4000 units, from about 12 units to about 3000 units, from about 25 units to about 2000 units, from about 50 units to about 1000 units, from about 100 units to about 500 units, or from about 125 units to about 250 units. The medium molecular weight heparin may be administered at a dose of about 18 unit/kg to about 5000 units/kg, for example from about 100 units/kg to about 4000 units per/kg, or from about 200 units/kg to about 800 units/kg. The medium molecular weight heparin may be administered at a dose of about 18 units/kg to about 75 units/kg. The dose may be given as a single dose or as a continuous dose. The dose may be given over a period of time. The period of time may be from about 1 month to about 12 months, for example from about 2 months to about 11 months, from about 3 months to about 10 months, from about 4 months to about 9 months, from about 5 months to about 8 months, from about 6 months to about 7 months. The period of time may be about 1 day to 7 days, about 2 days to about 6 days, about 3 days to about 5 days, about 4 days to about 5 days. The dose may be administered over about 1 hour to about 24 hours, about 2 hours to about 12 hours, about 3 hours to about 6 hours. The dose may be administered for the duration of the underlying endotheliopathy and raised VWF levels.

The medium molecular weight heparin may be administered at a dose of about 0.01 mg/kg to about 10 mg/kg, for example at a dose of about 0.05 mg/kg to about 9 mg/kg, about 0.5 mg/kg to about 8 mg/kg, about 1 mg/kg to about 7 mg/kg, about 1.5 mg/kg to about 6 mg/kg, or about 2 mg/kg to about 5 mg/kg. The dose may be given as a single dose or as a continuous dose. The dose may be given over a period of time. The period of time may be from about 1 month to about 12 months, for example from about 2 months to about 11 months, from about 3 months to about 10 months, from about 4 months to about 9 months, from about 5 months to about 8 months, from about 6 months to about 7 months. The period of time may be about 1 day to about 7 days, about 2 days to about 6 days, about 3 days to about 5 days, about 4 days to about 5 days. The dose may be administered over about 1 hour to about 24 hours, about 2 hours to about 12 hours, about 3 hours to about 6 hours. The dose may be administered for the duration of the underlying endotheliopathy and raised VWF levels.

The medium molecular weight heparin may be administered at a dose of from about 0.01 mg/kg to about 10 mg/kg, from about 0.05 mg/kg to about 8 mg/kg, from about 0.1 mg/kg to about 5 mg/kg, from about 0.5 mg/kg to about 2 mg/kg, from about 1 mg/kg to about 1.5 mg/kg. The dose may be given as a single dose or as a continuous dose. The dose may be given over a period of time. The period of time may be from about 1 month to about 12 months, for example from about 2 months to about 11 months, from about 3 months to about 10 months, from about 4 months to about 9 months, from about 5 months to about 8 months, from about 6 months to about 7 months. The period of time may be from about 1 day to about 7 days, from about 2 days to about 6 days, from about 3 days to about 5 days, from about 4 days to about 5 days. The dose may be administered over about 1 hour to about 24 hours, about 2 hours to about 12 hours, about 3 hours to about 6 hours. The dose may be administered for the duration of the underlying endotheliopathy and raised VWF levels.

The medium molecular weight heparin may be administered at a dose of from about 0.1 mg to about 5000 mg, from about 0.5 mg to about 2000 mg, from about 1 mg to about 1000 mg, from about 5 mg to about 900 mg, from about 10 mg to about 800 mg, from about 20 mg to about 700 mg, from about 30 mg to about 600 mg, from about 50 mg to about 500 mg, from about 75 mg to about 400 mg, from about 100 mg to about 300 mg, from about 125 mg to about 250 mg, from about 150 mg to about 200 mg. The dose may be given as a single dose or as a continuous dose. The dose may be given over a period of time. The period of time may be from about 1 month to about 12 months, for example from about 2 months to about 11 months, from about 3 months to about 10 months, from about 4 months to about 9 months, from about 5 months to about 8 months, from about 6 months to about 7 months. The period of time may be from about 1 day to about 7 days, from about 2 days to about 6 days, from about 3 days to about 5 days, from about 4 days to about 5 days. The dose may be administered over about 1 hour to about 24 hours, about 2 hours to about 12 hours, about 3 hours to about 6 hours. The dose may be administered for the duration of the underlying endotheliopathy and raised VWF levels.

The medium molecular weight heparin may be administered at a dose of, for example about 1 international units (IU), about 2 IU, about 5 IU, about 10 IU, about 15 IU, about 20 IU, about 25 IU, about 50 U, about 75 IU, about 100 U, about 200 U, about 300 U, about 400 U, about 500 U, about 1000 IU, about 1500 IU, about 2000 IU, about 2500 IU, about 5000 IU, about 10 000 IU, about 20 000 IU, or about 25 000 IU. The dose may be given as a single dose or as a continuous dose. The dose may be given over a period of time. The period of time may be from about 1 month to about 12 months, for example from about 2 months to about 11 months, from about 3 months to about 10 months, from about 4 months to about 9 months, from about 5 months to about 8 months, from about 6 months to about 7 months. The period of time may be from about 1 day to about 7 days, from about 2 days to about 6 days, from about 3 days to about 5 days, from about 4 days to about 5 days. The dose may be administered over about 1 hour to about 24 hours, about 2 hours to about 12 hours, about 3 hours to about 6 hours. The dose may be administered for the duration of the underlying endotheliopathy and raised VWF levels.

The medium molecular weight heparin may be administered at a dose of from about 1 IU to about 50 000 IU, from about 2 IU to about 25 000 IU, from about 5 IU to about 20 000 IU, from about 10 IU to about 10 000 IU, from about 15 IU to about 5000 IU, from about 20 IU to about 2500 IU, from about 25 IU to about 2000 IU, from about 50 IU to about 1500 IU, from about 75 IU to about 1000 IU, from about 100 IU to about 500 IU, from about 200 IU to about 400 IU, from about 250 IU to about 300 IU. The dose may be given as a single dose or as a continuous dose. The dose may be given over a period of time. The period of time may be from about 1 month to about 12 months, for example from about 2 months to about 11 months, from about 3 months to about 10 months, from about 4 months to about 9 months, from about 5 months to about 8 months, from about 6 months to about 7 months. The period of time may be from about 1 day to about 7 days, from about 2 days to about 6 days, from about 3 days to about 5 days, from about 4 days to about 5 days. The dose may be administered over about 1 hour to about 24 hours, about 2 hours to about 12 hours, about 3 hours to about 6 hours. The dose may be administered for the duration of the underlying endotheliopathy and raised VWF levels.

The medium molecular weight heparin may be administered that is commensurate with the VWF antigen: ADAMTS13 ratio. For example, a patient with a high VWF antigen: ADAMTS13 ratio may be administered a higher dose of MMWH compared to a patient with a VWF antigen: ADAMTS13 ratio that is lower.

The medium molecular weight heparin may be comprised in a pharmaceutical formulation. The pharmaceutical formulation comprises a composition of matter suitable for administration to a subject. The pharmaceutical formulation may be in liquid, solid, colloidal or aerosol form. The excipient may be selected from the group consisting of solvents, co-solvents, buffers, stabilisers, antioxidants, preservatives, chelating agents, emulsifiers, flavourings, lubricants, suspending agents, tonicity adjusting agents, surfactants, solubilising agents, suspending aids, dispersion agents, humectants, thickeners, colouring agent, wetting agent, anti-foaming agent, viscosity modifier, sweeteners and combinations thereof. The pharmaceutical formulation may comprise glucose. The pharmaceutical formulation may comprise sodium chloride. The pharmaceutical formulation may comprise phosphate buffered saline.

The pharmaceutical formulation may comprise an additional active agent. The additional active agent comprises a composition of matter that has a physiological effect. The additional active agent may comprise low molecular weight heparin or a medium molecular weight heparin of a different disaccharide composition. The additional active agent may be selected from the group comprising farnesoid X receptor (FXR) agonist, a peroxisome proliferator-activator receptor (PPAR) agonist, aramchol, a caspase inhibitor, a galectin 3 inhibitor, a mitogen-activated protein kinase 5 (MAPK5) inhibitor, a fibroblast growth factor 19 (FGF19) agonist, a FGF21 agonist, a leukotriene D4 (LTD4) receptor antagonist, a niacin analog, an apical sodium bile acid cotransporter (ASBT) inhibitor, an apoptosis signal regulating kinase 1 (ASK1) inhibitor, an angiotensin converting enzyme (ACE) inhibitor, an angiotensin receptor blocker, a chemokine receptor inhibitor, a thiozolidinedione, a GLP-1 analog, a biguanide, an HIV replication inhibitor, metoformin, an opiate, an anaesthetic, HMG-CoA reductase inhibitor, a nonsteroidal anti-inflammatory drug (NSAID), or any combination of these.

The medium molecular weight heparin may comprise a chemical modification. The chemical modification comprises any chemical change to the medium molecular weight heparin. Accordingly, the chemical change may comprise N-acetylation, N-deacetylation, N-sulfation, 0-sulfation, 2-0 desulfation, complete desulfation, or any combination of these.

The invention provides a kit comprising medium molecular weight heparin as defined herein for use in the treatment of endotheliopathy. The kit may comprise the medium molecular weight heparin in a unit dosage form, in a dosage as defined herein. The kit may comprise a pharmaceutical package. The kit may comprise the necessary reagents to synthesise the medium molecular weight heparin for use according to the invention.

The invention provides a method of treating endotheliopathy, the method comprising administering to a subject in need of treatment a therapeutically effective amount of medium molecular weight heparin as defined herein. The therapeutic effective amount is any amount of medium molecular weight heparin required to treat to some extent the endotheliopathy.

The invention provides the use of the medium molecular weight heparin as defined herein for the manufacture of a medicament for the treatment of endotheliopathy.

Every document cited herein, including any cross-referenced or related patent or application, is hereby incorporated by reference in its entirety unless expressly excluded or otherwise limited.

It will be appreciated that various modifications may be made to the embodiments shown without departing from the spirit and scope of the invention as defined by the accompanying claims.

### Examples

The invention will now be demonstrated by reference to the following non-limiting examples.

Unless otherwise mentioned, room temperature and pressure are 20° C. (293.15 K, 68° F.) and 1 atm (14.696 psi, 101.325 kPa), respectively.

### Dissaccharide Analysis

Disaccharide analysis was carried out on the MMW heparin. The results are shown in Table 1 below.

| | % Composition MMW Heparin. |
|---|---|
| Disaccharides | NOTE: only ~60% breakdown |
| UA-GlcNAc | 9.0 |
| UA-GlcNS | — |
| UA-GlcNAc6S | — |
| UA2S-GlcNAc | — |
| UA-GlcNS6S | — |
| UA2S-GlcNS | 9.0 |
| UA2S-GlcNAc6S | — |
| UA2S-GlcNS6S | 82.0 |

Factor IIa and Factor Xa activity

Factor IIa (also known as thrombin) acts as a serine protease that converts soluble fibrogen into insoluble strands of fibrin, as well as catalysing other coagulation-related reactions. Factor Xa is the activated form of the coagulation factor X. Factor X is an serine endopeptidase enzyme, which plays a key role at several stages of the coagulation system.

Heparin (unfractionated heparin) and its derivatives, e.g. low molecular weight heparin, bind to a plasma cofactor, antithrombin (AT), to inactivate several coagulation factors IIa, Xa, XIa and XIIa. This inactivation of Factor Xa by heparins is termed “indirect” since it relies on the presence of AT and not a direct interaction with Factor Xa.

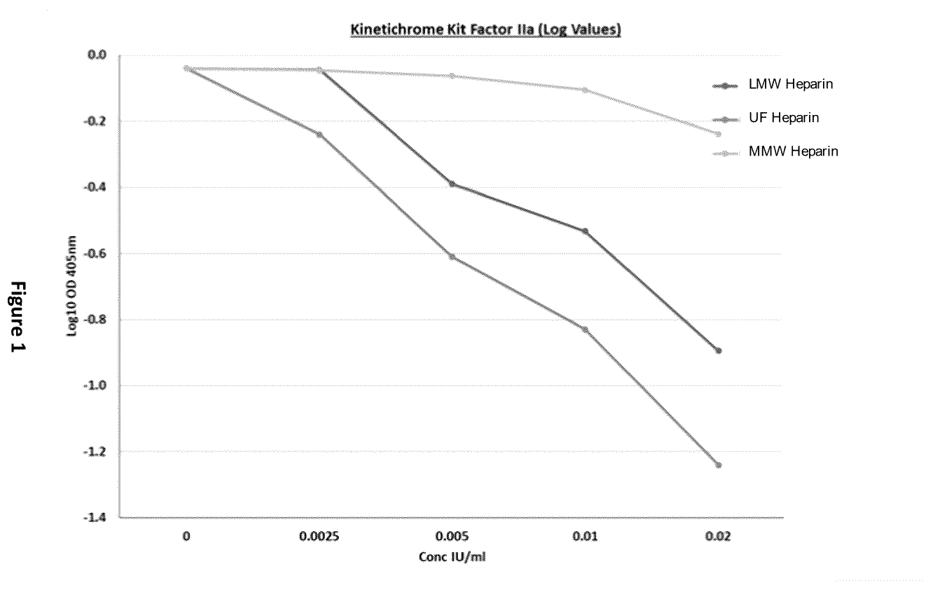

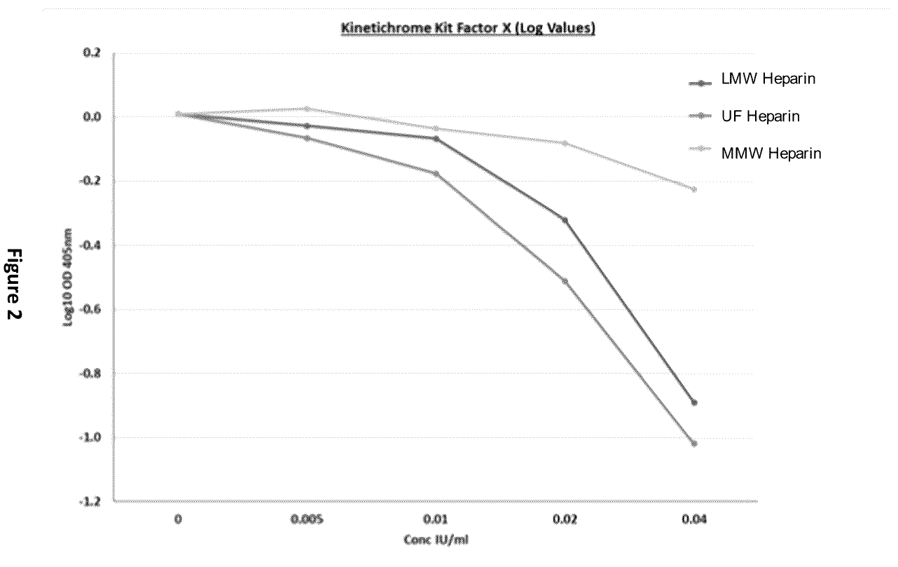

As shown in FIGS. 1 and 2, MMW Heparin exhibits very low activity against Factor IIa and Factor Xa, respectively, as compared to UF Heparin and LMW (low molecular weight) Heparin. Thus, unlike UF Heparin or LMW Heparin, MMW Heparin does not affect Factor IIa or Factor Xa-mediated coagulation.

### Analysis of MMW Heparin in a Ristocetin-Induced Platelet Aggregation Assay

Protocol according to “Recommendations for the standardization of light transmission aggregometry: a consensus of the working party from the platelet physiology subcommittee of SSC/ISTH”, Journal of Thrombosis and Haemostasis, 2013, 11: 1183-1189.

### Preparation of the Ristocetin Working Solution:

Ristocetin stock (50 mg/mL) was diluted with saline to 24 mg/mL. 20 μL of 24 mg/mL ristocetin in saline in a 400 μL solution provides a solution with a ristocetin concentration of 1.2 mg/mL.

### Preparation of the MMW Heparin Working Solutions:

MMW heparin (11 kDa, 0,569 g) was dissolved in H₂O (10 mL) to provide a 5.2 mM solution of MMW heparin in H₂O. The 5.2 mM stock solution of MMW heparin in H₂O was frozen at −20° C.

MMW heparin was diluted in saline as follows:

| | Volume | | | | |
|---|---|---|---|---|---|
| MMW | MMW | Volume | | Final MMW heparin | |
| heparin | heparin | of MMW | | concentration in | |
| concen- | stock | heparin | Volume | aggregation test | |
| tration | (5.2 mM) | diluted | of saline | (20 μL in 400 μL) | |
| 300 μM | 57.6 μL | | 942 μL | 15 | μM |
| 200 μM | | 667 μL of | 333 μL | 10 | μM |
| | | 300 μM | | | |
| 100 μM | | 300 μL of | 300 μL | 5 | μM |
| | | 200 μM | | | |

### Protocol

Blood samples were taken from a non-smoker not on any anti-platelet therapy (for example, aspirin). An anti-platelet therapy may also be known as a platelet agglutination inhibitor or a platelet aggregation inhibitor.

Blood was drawn, with no venostasis, from a donor into 109 mM sodium citrate solution (VACUETTE, 3.5 mL #454327, lot #A21013FQ). The first 3 to 4 mL of blood drawn was discarded. Blood samples were allowed to ‘rest’ at room temperature for 15 min before centrifugation. Platelet rich plasma (PRP) was prepared by centrifuging blood samples at 200 g for 10 min at 21° C., without using brake. Platelet poor plasma (PPP) was prepared by centrifuging blood samples, from which PRP was removed, at 1500 g for 15 min at 21° C.

An assessment of PRP quality was made by carrying out a platelet count of the PRP. Platelet count in PRP was 421 G/L. The platelet count of PRP samples was not (and should not be) adjusted to a standardised value with autologous PPP.

After centrifugation, PRP samples were allowed to sit at room temperature for 15 min before Light Transmission Aggregometry (LTA) studies were carried out. PRP was used to set 0% light transmission in the aggregometer. Autologous PPP was used to set 100% light transmission in the aggregometer.

The following steps were carried out for each of the three MMW heparin working solution concentrations prepared (5 μM, 10 μM and 15 M).

20 μL of MMW heparin diluted in saline (described above under Preparation of the MMW heparin working solutions) was added to 360 μL of PRP. The resultant solution was stirred for 2 seconds and then incubated without stirring at 37° C. for 5 minutes. The solution was then incubated with agitation at 37° C. for 1 minute. Prior to adding the agonist (ristocetin), baseline tracings for LTA were observed for oscillations and stability for at least 1 minute.

20 μL of the ristocetin working solution, i.e. ristocetin diluted in saline (as described above under Preparation of the ristocetin working solution) was added to the MMW heparin and PRP solution to give a final solution volume of 400 μL.

LTA studies were performed at 37° C. During the LTA studies, the PRP samples were constantly stirred at 1000 rpm using a disposable stirrer.

The volume of agonist (ristocetin) added for the LTA studies should be consistent, and not more than 10% of the total sample volume. In the present example, the volume of agonist should not be more than 40 μL.

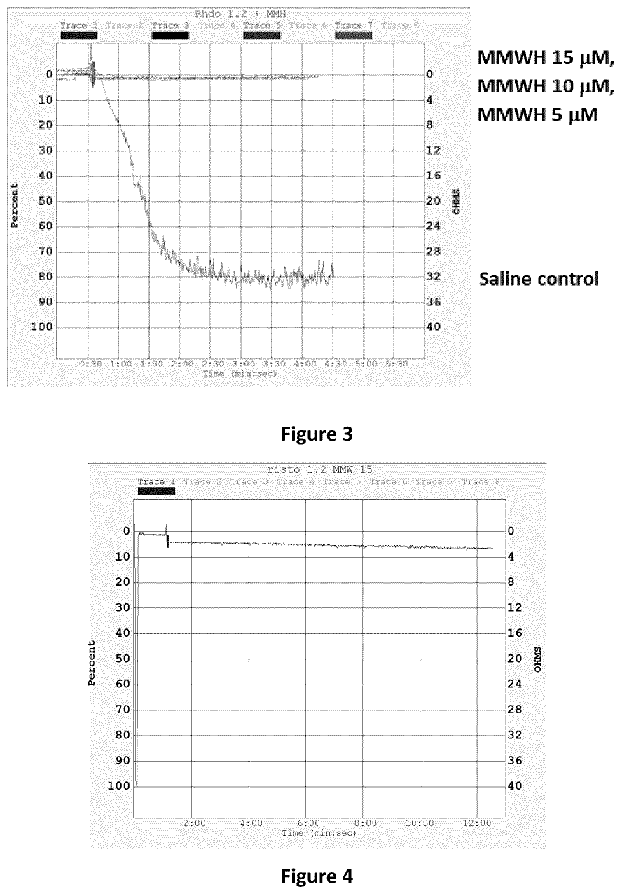

Three doses of MMW heparin were tested: 5 μM, 10 μM and 15 μM, which were compared to the saline vehicle control. As shown in FIG. 3, all of the tested concentrations (5 μM, 10 μM and 15 μM) fully inhibited VWF-induced platelet aggregation.

The highest MMW heparin concentration, 15 μM, was re-tested. This retest gave a similar result, i.e. full inhibition of VWF-induced platelet aggregation, as shown in FIG. 4.

Thus, complete inhibition of VWF-induced platelet aggregation was observed when adding MMW heparin to PRP samples.

### Dose-Dependent MMWH Inhibition of Von Willebrand Factor-Platelet Binding

Protocol according to “Recommendations for the standardization of light transmission aggregometry: a consensus of the working party from the platelet physiology subcommittee of SSC/ISTH”, Journal of Thrombosis and Haemostasis, 2013, 11: 1183-1189.

### Preparation of the Ristocetin Working Solution:

Ristocetin stock (50 mg/mL) was diluted with saline to 48 mg/mL.

### Preparation of the MMW Heparin Working Solutions:

MMW heparin (11 kDa, 0,569 g) was dissolved in H₂O (10 mL) to provide a 5.2 mM solution of MMW heparin in H₂O. The 5.2 mM stock solution of MMW heparin in H₂O was frozen at −20° C.

20 μL was used in each aggregation test, corresponding to a dilution of 20 times. MMWH was diluted in saline as follows:

| | |
|---|---|
| Volume | Final MMWH |
| MMWH | concentration in |

| | | | |
|---|---|---|---|
| MMW heparin | stock | Volume | aggregation test |
| concentration | (5.2 mM) | of saline | (20 μL in 400 μL) |

| | | | | | | |
|---|---|---|---|---|---|---|
| 400 μM | 39 | μL | 431 | μL | 20 | μM |
| 200 μM | 19 | μL | 481 | μL | 10 | μM |
| 100 μM | 9.5 | μL | 490.5 | μL | 5 | μM |

### Monoclonal Anti-VWF

As a positive control, an anti-VWF monoclonal antibody which blocks ristocetin induced platelet aggregation was used. Briefly, Ab #701 5.5 mg/mL was diluted in saline at 200 μg/mL. 20 μL of the 200 μg/mL Ab #701 in saline was further diluted with 400 μL PRP to give a final antibody concentration of 10 μg/mL.

### Preparation of LMWH: Lovenox (Enoxiparin) Solutions

Stock 8000 UI: 100 mg/mL; Molecular weight: 4500 Da; Lovenox Stock concentration: 22 mM

To prepare a final concentration of 20 μM LMWH: dilute 18 μL Lovenox 22 mM stock in 1000 μL saline to give a 400 μM stock solution. 20 μL of the 400 μM stock solution in a final volume of 400 μL provides a final concentration of LMWH of 20 M.

To prepare a final concentration of 10 μM LMWH: dilute 9 μL Lovenox 22 mM stock in 1000 μL saline to give a 200 μM stock solution. 20 μL of the 200 μM stock solution in a final volume of 400 μL provides a final concentration of LMWH of 10 M.

### Agonist-Induced Aggregation

ADP: Stock concentration 5 mM, diluted at 40 μM in saline. 20 μL of the 40 μM ADP stock solution in a final volume of 400 μL provides a final concentration of ADP of 2 μM.

Collagen: Stock concentration 1 mg/mL, diluted at 40 μg/mL in saline. 20 μL of the 40 μg/mL Collagen stock solution in a final volume of 400 μL provides a final concentration of Collagen of 2 μg/mL.

TRAP6: Stock concentration 20 mM, diluted at 200 μM in saline. 20 μL of the 200 μM ADP stock solution in a final volume of 400 μL provides a final concentration of ADP of 10 μM.

### Protocol

Blood samples were taken from a non-smoker not on any anti-platelet therapy (for example, aspirin).

Blood was drawn, with no venostasis, from a donor into 109 mM sodium citrate solution (VACUETTE, 3.5 mL #454327, lot #A21013FQ). The first 3 to 4 mL of blood drawn was discarded.

Blood samples were allowed to ‘rest’ at room temperature for 15 min before centrifugation. Platelet rich plasma (PRP) was prepared by centrifuging blood samples at 200 g for 10 min at 21° C., without using brake. Platelet poor plasma (PPP) was prepared by centrifuging blood samples, from which PRP was removed, at 1500 g for 15 min at 21° C.

An assessment of PRP quality was made by carrying out a platelet count of the PRP. The platelet count of PRP samples was not (and should not be) adjusted to a standardised value with autologous PPP.

After centrifugation, PRP samples were allowed to sit at room temperature for 15 min before Light Transmission Aggregometry (LTA) studies. PRP was used to set 0% light transmission in the aggregometer. Autologous PPP was used to set 100% light transmission in the aggregometer. LTA studies were performed at 37° C. During the LTA studies, the PRP samples were constantly stirred at 1000 rpm using a disposable stirrer. The volume of agonist added for LTA should be consistent, and never more than 10% of the total sample volume

### MMWH Test Inhibition

20 μL MMWH (5, 10 or 20 μM) and 10 μL ReoPro (final concentration of 20 μg/mL) were added to 360 μL PRP. The resultant solution was stirred for 2 seconds and then incubated without stirring at 37° C. for 5 minutes. The solution was then incubated with agitation at 37° C. for 1 minute. Prior to adding the agonist (ristocetin), baseline tracings for LTA were observed for oscillations and stability for at least 1 minute.

10 μL of the ristocetin working solution, i.e. ristocetin diluted in saline (as described above under Preparation of the ristocetin working solution) was added to the MMWH, ReoPro and PRP solution to give a final solution volume of 400 μL, and a final ristocetin concentration of 1.2 mg/mL.

A control experiment using 20 μL saline in place of MMWH in the method above was also carried out.

### LMWH Test Inhibition

20 μL LMWH (10 or 20 μM) and 10 μL ReoPro (final concentration of 20 μg/mL) were added to 360 μL PRP. The resultant solution was stirred for 2 seconds and then incubated without stirring at 37° C. for 5 minutes. The solution was then incubated with agitation at 37° C. for 1 minute. Prior to adding the agonist (ristocetin), baseline tracings for LTA were observed for oscillations and stability for at least 1 minute.

10 μL of the ristocetin working solution, i.e. ristocetin diluted in saline (as described above under Preparation of the ristocetin working solution) was added to the LMWH, ReoPro and PRP solution to give a final solution volume of 400 μL, and a final ristocetin concentration of 1.2 mg/mL.

A control experiment using 10 μL saline in place of ReoPro in the method above was also carried out.

### Monoclonal Anti-VWF Test Inhibition

20 μL Ab #701 and 10 μL ReoPro (final concentration of 20 μg/mL) were added to 360 μL PRP. The resultant solution was stirred for 2 seconds and then incubated without stirring at 37° C. for 5 minutes. The solution was then incubated with agitation at 37° C. for 1 minute. Prior to adding the agonist (ristocetin), baseline tracings for LTA were observed for oscillations and stability for at least 1 minute.

10 μL of the ristocetin working solution, i.e. ristocetin diluted in saline (as described above under Preparation of the ristocetin working solution) was added to the Monoclonal anti-VWF, ReoPro and PRP solution to give a final solution volume of 400 IL, and a final ristocetin concentration of 1.2 mg/mL.

### MMWH Test Inhibition in Agonist-Induced Aggregation

Agonists investigated were ADP, Collagen and TRAP6.

20 μL MMWH (5, 10 or 20 μM) or 20 μL saline were added to 360 μL PRP. The resultant solution was stirred for 2 seconds and then incubated without stirring at 37° C. for 5 minutes. The solution was then incubated with agitation at 37° C. for 1 minute. Prior to adding the agonist, baseline tracings for LTA were observed for oscillations and stability for at least 1 minute.

20 μL agonist (ADP, Collagen or TRAP6) was added to the MMWH and PRP solution to give a final solution volume of 400 μL, and a final agonist concentration as follows:
ADP: 2 μM;
Collagen: 2 μg/mL; or
TRAP6: 10 μM

### Results

The experiments were performed in the presence of ReoPro (inhibitor of αIIbβ3) to analyse the agglutination phase selectively. Outcomes are shown as slope and area under the curve (AUC). 1) MMWH dose responses (0, 5, 10 and 20 μM)

| | Control | MMWH 5 μM | MMWH 10 μM | MMWH 20 μM |
|---|---|---|---|---|
| Slope | 81 | 28 | 6 | 2 |
| AUC | 23.2 | 7.6 | 1 | 0.2 |

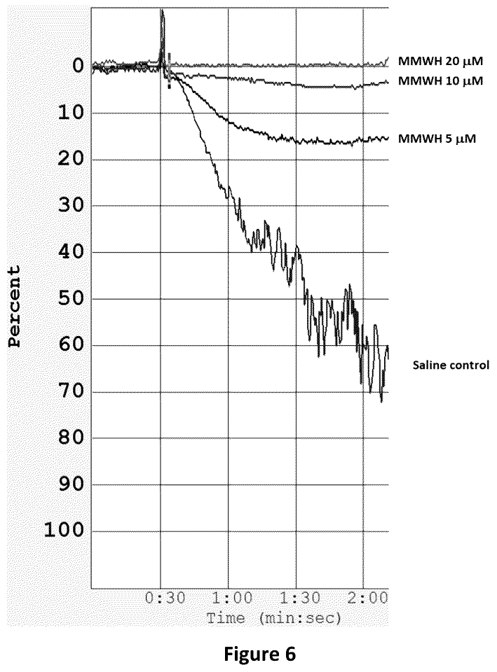

The dose response curve is shown in FIG. 6.

### 2) MMWH Dose Responses (0, 5 and 10 μM)

| | Control | MMWH 5 μM | MMWH 10 μM |
|---|---|---|---|
| Slope | 74 | 20 | 4 |
| AUC | 22 | 4.8 | 0.2 |

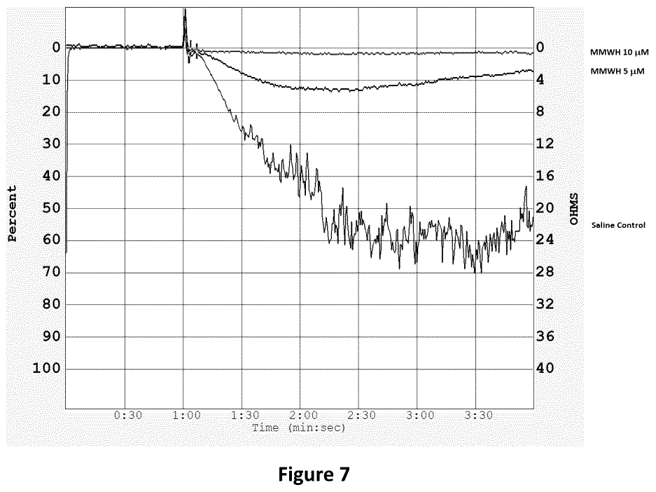

The dose response curve is shown in FIG. 7.

### 3) mAb and MMWH 10 μM

| | Control | MMWH 10 μM | mAb |
|---|---|---|---|
| Slope | 79 | 6 | 4 |
| AUC | 27.8 | 1.2 | 0.3 |

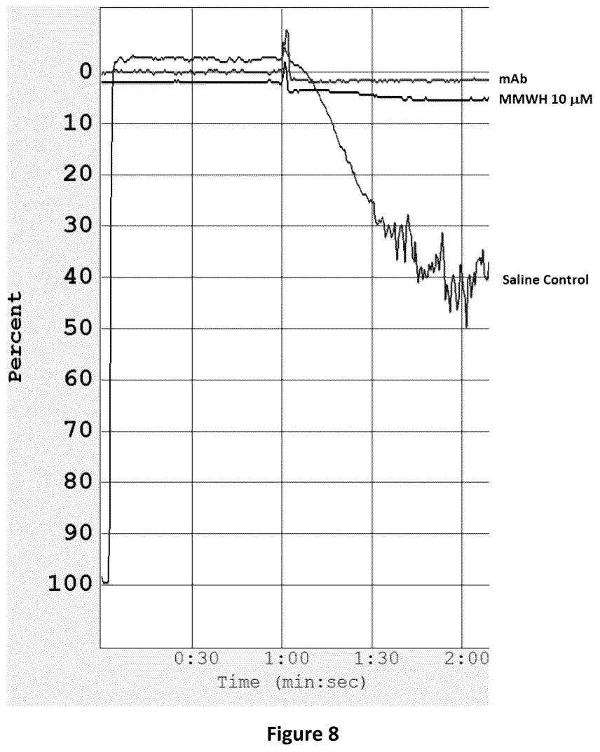

The dose response curve is shown in FIG. 8.

### 4) mAb (Anti-VWF) and MMWH (20 μM×2 and 5 μM) Dose Responses

| | mAb | MMWH 5 μM | MMWH 20 μM | MMWH 20 μM |
|---|---|---|---|---|
| Slope | 4 | 44 | 2 | 4 |
| AUC | 0.2 | 13.6 | 0 | 0 |

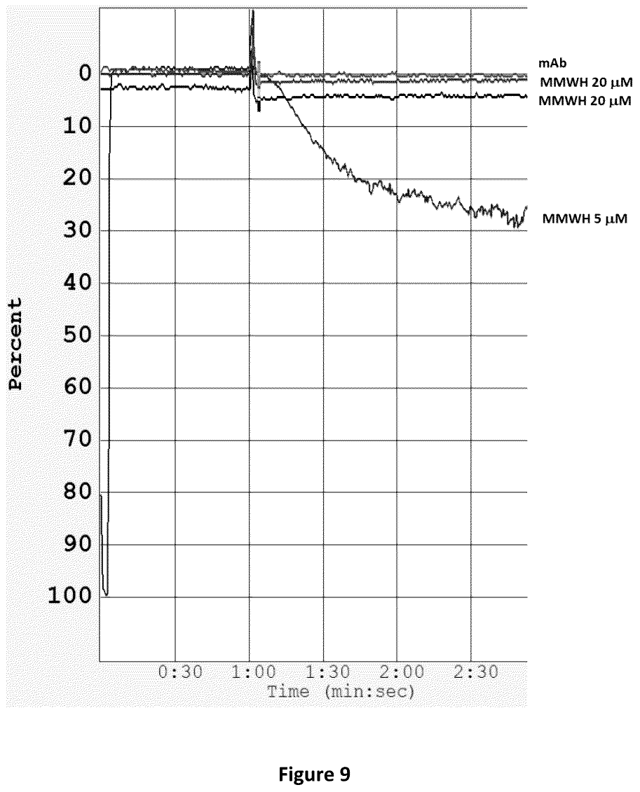

The dose response curve is shown in FIG. 9.

### 5) mAb (Anti-VWF) and LMWH (10 μM×3) Dose Responses

| | mAb | LMWH 10 μM | LMWH 10 μM | LMWH 10 μM |
|---|---|---|---|---|
| Slope | 2 | 68 | 61 | 72 |
| AUC | 0.1 | 19.3 | 17.4 | 19.4 |

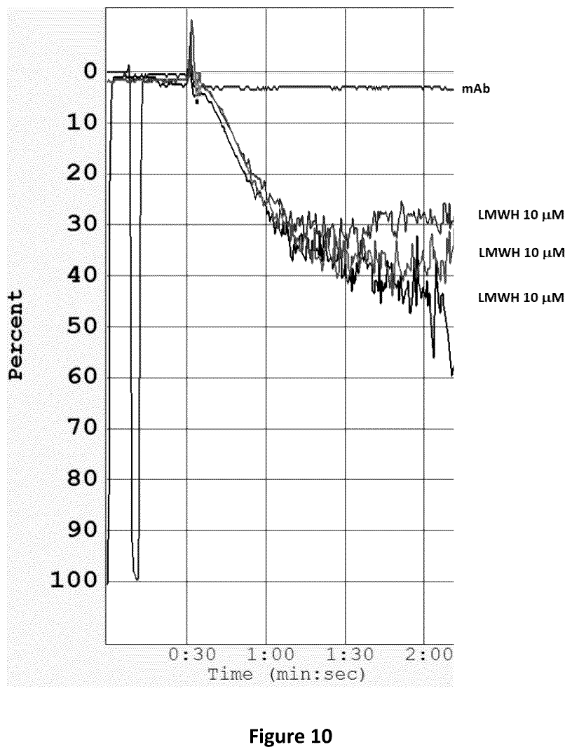

The dose response curve is shown in FIG. 10.

6) LMWH (20 μM×3) dose response

| | LMWH 20 μM | LMWH 20 μM | LMWH 20 μM |
|---|---|---|---|
| Slope | 63 | 53 | 55 |
| AUC | 18.6 | 15.1 | 14.5 |

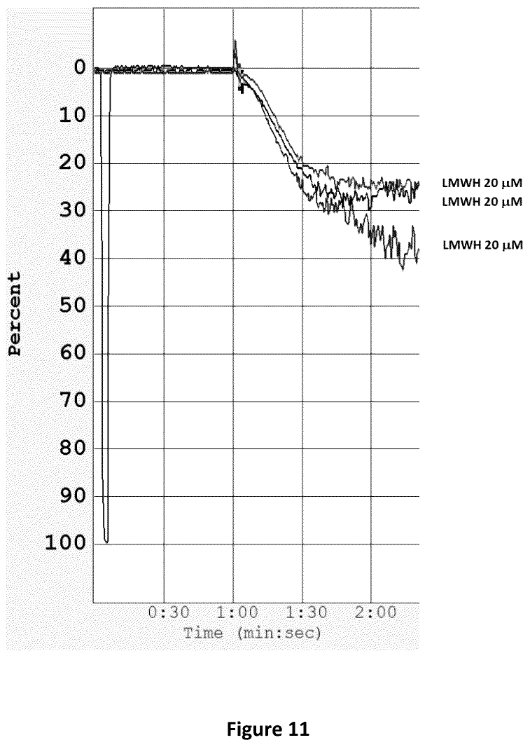

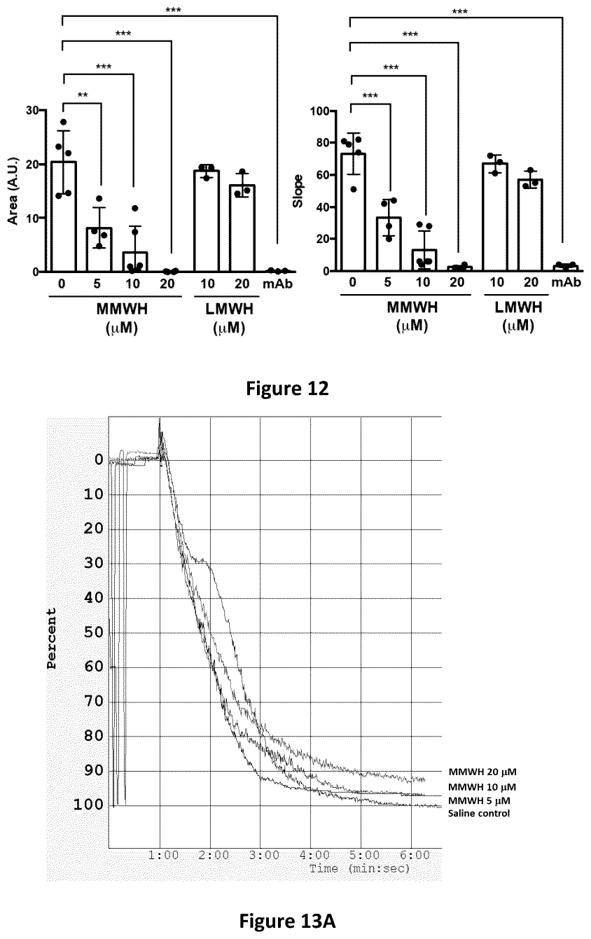

The dose response curve is shown in FIG. 11.

### 7) Statistical Analysis

| Table Analysed | Area |
|---|---|
| ANOVA summary | |
| F | 20.28 |
| P value | <0.0001 |
| P value summary | **** |
| Are differences among means | Yes |
| statistically significant? (P < 0.05) | |
| R square | 0.8528 |
| Brown-Forsythe test | |
| F (DFn, DFd) | 1.362 (6, 21) |
| P value | 0.2750 |
| P value summary | ns |
| Significantly different standard | No |
| deviations? (P < 0.05) | |

| ANOVA table | SS | DF | MS | F (DFn, DFd) | P value |
|---|---|---|---|---|---|
| Treatment | 1796 | 6 | 299.3 | F (6, 21) = | P < 0.0001 |
| (between | | | | 20.28 | |
| columns) | | | | | |
| Residual | 309.9 | 21 | 14.76 | | |
| (within | | | | | |
| columns) | | | | | |
| Total | 2106 | 27 | | | |
| Data summary | | | | | |
| Number of | 7 | | | | |
| treatments | | | | | |
| (columns) | | | | | |
| Number of | 28 | | | | |
| values (total) | | | | | |

| Turkey's multiple | Mean | | Signif- | |
|---|---|---|---|---|
| comparisons test | Diff. | 95% CI of diff. | icant? | Summary |
| Control vs | 12.14 | 3.763 to 20.52 | Yes | ** |
| MMWH 5 | | | | |
| Control vs | 16.67 | 9.112 to 24.23 | Yes | **** |
| MMWH 10 | | | | |
| Control vs | 20.27 | 11.89 to 28.64 | Yes | **** |
| MMWH 20 | | | | |
| Control vs | 20.14 | 11.02 to 29.26 | Yes | **** |
| mAb | | | | |
| Control vs | 1.640 | −7.480 to 10.76 | No | ns |
| LMWH 10 | | | | |
| Control vs | 4.273 | −4.846 to 13.39 | No | ns |
| LMWH 20 | | | | |

### One-Way ANOVA (Slope)

| Table Analysed | Slope |
|---|---|
| ANOVA summary | |
| F | 40.68 |
| P value | <0.0001 |
| P value summary | **** |
| Are differences among means | Yes |
| statistically significant? (P < 0.05) | |
| R square | 0.9208 |
| Brown-Forsythe test | |
| F (DFn, DFd) | 0.8456 (6, 21) |
| P value | 0.5493 |
| P value summary | ns |
| Significantly different standard | No |
| deviations? (P < 0.05) | |

| ANOVA table | SS | DF | MS | F (DFn, DFd) | P value |
|---|---|---|---|---|---|
| Treatment | 21997 | 6 | 3666 | F (6, 21) = | P < 0.0001 |
| (between | | | | 40.68 | |
| columns) | | | | | |
| Residual | 1893 | 21 | 90.13 | | |
| (within | | | | | |
| columns) | | | | | |
| Total | 23890 | 27 | | | |
| Data summary | | | | | |
| Number of | 7 | | | | |
| treatments | | | | | |
| (columns) | | | | | |
| Number of | 28 | | | | |
| values (total) | | | | | |

| Turkey's multiple | Mean | | Signif- | |
|---|---|---|---|---|
| comparisons test | Diff. | 95% CI of diff. | icant? | Summary |
| Control vs | 39.90 | 19.20 to 60.60 | Yes | **** |
| MMWH 5 | | | | |
| Control vs | 60.23 | 41.55 to 78.92 | Yes | **** |
| MMWH 10 | | | | |
| Control vs | 70.90 | 50.20 to 91.60 | Yes | **** |
| MMWH 20 | | | | |
| Control vs | 70.07 | 47.53 to 92.60 | Yes | **** |
| mAb | | | | |
| Control vs | 6.400 | −1614 to 28.94 | No | ns |
| LMWH 10 | | | | |
| Control vs | 16.40 | −6.138 to 38.94 | No | ns |
| LMWH 20 | | | | |

### 8) Agonist-Induced Platelet Aggregation

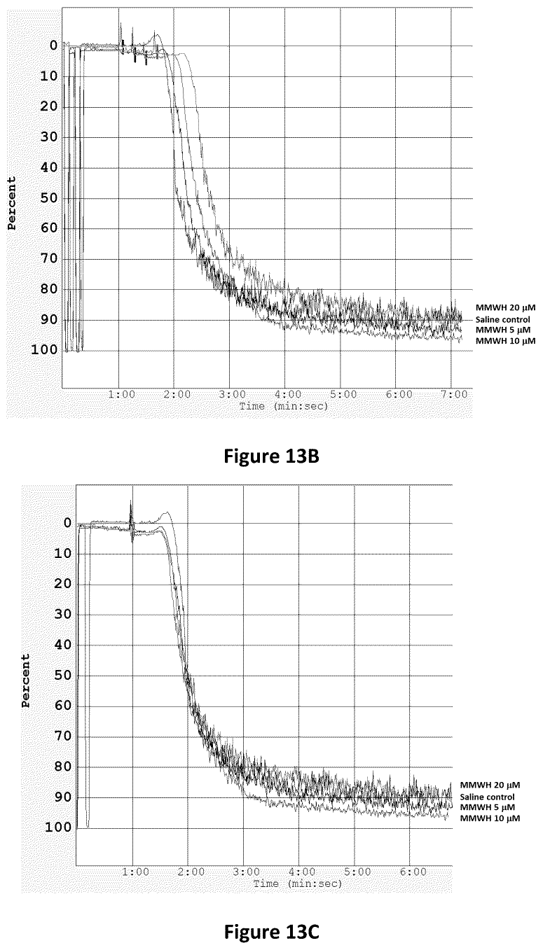

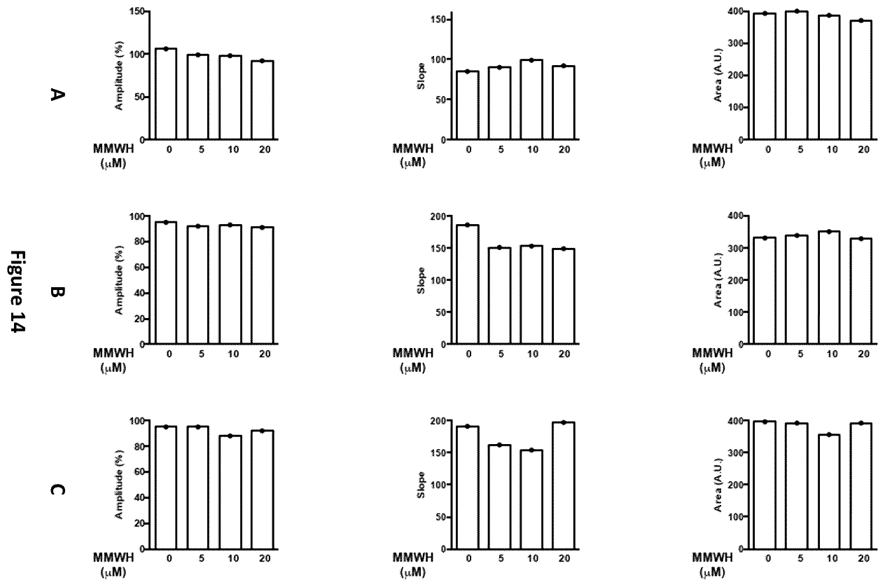

As shown in FIGS. 13 and 14, no effect on platelet aggregation was observed when ADP, Collagen or TRAP6 were used as agonist.

### CONCLUSION

MMWH has been tested in von Willebrand factor (VWF)-dependent platelet agglutination experiments as well as in platelet aggregation tests induced by ADP, collagen or Thrombin Receptor Activating Peptide-6 (TRAP6). In platelet-agglutination experiments, low molecular weight heparin (LMWH) and a monoclonal anti-VWF antibody were used as a negative and positive control, respectively.

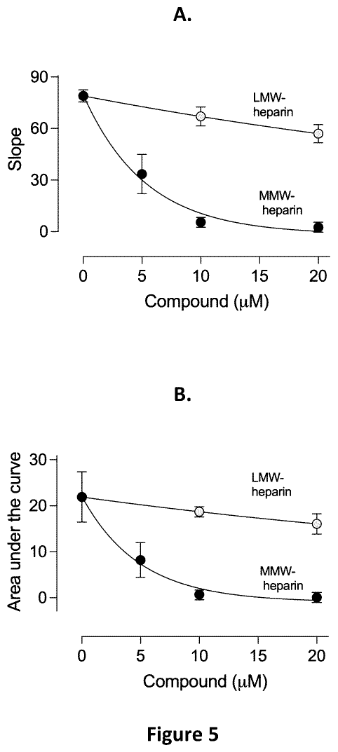

As shown in FIGS. 5A and 51B, VWF-dependent platelet agglutination was efficiently inhibited in a dose-dependent manner by MMWH, no significant inhibition was observed in the presence of LMWH. In contrast, the anti-VWF monoclonal antibody (known to interfere with VWF-platelet interactions) fully inhibited platelet agglutination. Half-maximal inhibition was obtained at a MMWH concentration of 3.4-3.7 μM.

As shown in FIGS. 13 and 14, no effect of platelet aggregation was observed when other agonists were used.

The invention may be further understood with reference to the following non-limiting clauses:

1. Medium molecular weight heparin for use in the treatment of endotheliopathy.

2. Medium molecular weight heparin for the use according to clause 1, wherein the medium molecular weight heparin inhibits von Willebrand factor.

3. Medium molecular weight heparin for the use according to clause 2, wherein the medium molecular weight heparin inhibits multimers of von Willebrand factor, optionally wherein the von Willebrand factor is ultra-large von Willebrand factor.

4. Medium molecular weight heparin for the use according to any of the preceding clauses, wherein medium molecular weight heparin inhibits the binding of platelets to von Willebrand factor.

5. Medium molecular weight heparin for the use according to any of the preceding clauses, wherein the medium molecular weight heparin has a mass in the range of greater than about 8000 Da (g/mol) to about 13 000 Da (g/mol), optionally wherein the medium molecular weight heparin a mass of about 11 000 Da (g/mol).

6. Medium molecular weight heparin for the use according to any of the preceding clauses, wherein the medium molecular weight heparin comprises at least three units of a IdoA2S-GlcNS6S disaccharide.

7. Medium molecular weight heparin for the use according to any of the preceding clauses, wherein the endotheliopathy is caused by COVID-19, viral infection, acute respiratory distress syndrome, cancer, infection, septicaemia, cardiovascular disease, diabetes mellitus, trauma, in particular brain or head trauma, burns, inhalational injury, drugs and drug reactions, haematological conditions, subarachnoid haemorrhage, aneurysmal diseases, stroke, or brain parenchymal haemorrhage.

8. Medium molecular weight heparin for the use according to clause 7 wherein the endotheliopathy is caused by viral infection, optionally wherein the viral infection is SARS-CoV-2.

9. Medium molecular weight heparin for the use according to clause 7 wherein the endotheliopathy is caused by cancer, optionally wherein the cancer is leukaemia, lymphoma, myeloma, or a solid organ cancer.

10. Medium molecular weight heparin for the use according to clauses 1 to 6 wherein the treatment of endotheliopathy inhibits the haematogenous spread of cancer.

11. Medium molecular weight heparin for the use according to any of the preceding clauses, wherein the medium molecular weight heparin is administered by an administration method selected from the group consisting of: parenteral, subcutaneous, depot form, for example depot injection, intravenous, intramuscular, intrathecal, intradermal, intraarterial, intraarticular, cutaneous, transcutaneous, intra-osseus, or inhalation.

12. Medium molecular weight heparin for the use according to clause 11, wherein the administration method is subcutaneous.

13. Medium molecular weight heparin for the use according to clause 11, wherein the administration method is intravenous.

14. Medium molecular weight heparin for the use according to clause 11, wherein the administration method is intramuscular.

15. Medium molecular weight heparin for the use according to clause 11, wherein the administration method is inhalation, optionally via a nebuliser.

16. Medium molecular weight heparin for the use according to any of the preceding clauses, wherein the Medium molecular heparin is administered at a dose of about 0.01 mg/kg to about 10 mg/kg.

17. Medium molecular weight heparin for the use according to any one of clauses 11 to 16, wherein the medium molecular weight heparin is administered as a single dose or a continuous dose.

18. Medium molecular weight for the use according to any of the preceding clauses, wherein the medium molecular weight heparin is comprised in a pharmaceutical formulation.

19. Medium molecular weight for the use according to clause 18, wherein the pharmaceutical formulation comprises an excipient.

20. Medium molecular weight for the use according to clause 19, wherein the excipient is selected from the group consisting of solvents, co-solvents, buffers, stabilisers, antioxidants, preservatives, chelating agents, emulsifiers, flavourings, lubricants, suspending agents, tonicity adjusting agents, surfactants, solubilising agents, suspending aids, dispersion agents, humectants, thickeners, colouring agent, wetting agent, anti-foaming agent, viscosity modifier, sweeteners and combinations thereof.

21. Medium molecular weight heparin for the use according to any one of clauses 18 to 20, wherein the pharmaceutical formulation comprises an additional active agent, optionally wherein the additional active agent comprises low molecular weight heparin or a medium molecular weight heparin of a different disaccharide composition.

22. Medium molecular weight heparin for the use according to any of the preceding clauses, wherein the medium molecular weight heparin comprises a chemical modification.

23. Medium molecular weight heparin for the use according to clause 22 wherein the chemical modification comprises N-acetylation, N-deacetylation, N-sulfation, 0-sulfation, 2-0 desulfation, complete desulfation, or combinations thereof.

24. A kit comprising a medium molecular weight heparin for use according to clauses 1-23.

25. A method of treating endotheliopathy, the method comprising administering to a subject in need of treatment a therapeutically effective amount of medium molecular weight heparin.

### REFERENCES

1. Zhu N, Zhang D, Wang W, Li X, Yang B, Song J, et al. A Novel Coronavirus from Patients with Pneumonia in China, 2019. N Engl J Med. 2020 20; 382(8):727-33.
2. Chen N, Zhou M, Dong X, Qu J, Gong F, Han Y, et al. Epidemiological and clinical characteristics of 99 cases of 2019 novel coronavirus pneumonia in Wuhan, China: a descriptive study. Lancet Lond Engl. 2020 15; 395(10223):507-13.
3. Huang C, Wang Y, Li X, Ren L, Zhao J, Hu Y, et al. Clinical features of patients infected with 2019 novel coronavirus in Wuhan, China. Lancet Lond Engl. 2020 15; 395(10223):497-506.
4. Wang D, Hu B, Hu C, Zhu F, Liu X, Zhang J, et al. Clinical Characteristics of 138 Hospitalized Patients With 2019 Novel Coronavirus-Infected Pneumonia in Wuhan, China. JAMA. 2020 Feb. 7;
5. Ñamendys-Silva S A. Respiratory support for patients with COVID-19 infection. Lancet Respir Med. 2020; 8(4):e18.
6. Cohen J, Vincent J-L, Adhikari N K J, Machado F R, Angus D C, Calandra T, et al. Sepsis: a roadmap for future research. Lancet Infect Dis. 2015 May; 15(5):581-614.
7. Angus D C, van der Poll T. Severe sepsis and septic shock. N Engl J Med. 2013 Aug. 29; 369(9):840-51.
8. Johansen M E, Johansson P I, Ostrowski S R, Bestle M H, Hein L, Jensen A L G, et al. Profound endothelial damage predicts impending organ failure and death in sepsis. Semin Thromb Hemost. 2015 February; 41(1):16-25.
9. Ostrowski S R, Haase N, Müller R B, Moller M H, Pott F C, Perner A, et al. Association between biomarkers of endothelial injury and hypocoagulability in patients with severe sepsis: a prospective study. Crit Care Lond Engl. 2015 Apr. 24; 19:191.
10. Varga Z, Flammer A J, Steiger P, Haberecker M, Andermatt R, Zinkernagel A S, et al. Endothelial cell infection and endotheliitis in COVID-19. The Lancet [Internet]. 2020 April 20 [cited 2020 Apr. 20]; 0(0). Available from: https://www.thelancet.com/journals/lancet/article/PIIS0140-6736(20)30937-5/abstract
11. Chang J C. Hemostasis based on a novel “two-path unifying theory” and classification of hemostatic disorders. Blood Coagul Fibrinolysis Int J Haemost Thromb. 2018 November; 29(7):573-84.
12. Chang J C. TTP-like syndrome: novel concept and molecular pathogenesis of endotheliopathy-associated vascular microthrombotic disease. Thromb J. 2018; 16:20.
13. Chang J C. Thrombogenesis and thrombotic disorders based on “two-path unifying theory of hemostasis”: philosophical, physiological, and phenotypical interpretation. Blood Coagul Fibrinolysis Int J Haemost Thromb. 2018 November; 29(7):585-95.
14. Chang J C. Disseminated intravascular coagulation: is it fact or fancy? Blood Coagul Fibrinolysis Int J Haemost Thromb. 2018 April; 29(3):330-7.
15. Chang J C. Sepsis and septic shock: endothelial molecular pathogenesis associated with vascular microthrombotic disease. Thromb J. 2019; 17:10.
16. Li T S, Gomersall C D, Joynt G M, Chan DPS, Leung P, Hui D S C. Long-term outcome of acute respiratory distress syndrome caused by severe acute respiratory syndrome (SARS): an observational study. Crit Care Resusc J Australas Acad Crit Care Med. 2006 December; 8(4):302-8.
17. Gralinski L E, Baric R S. Molecular pathology of emerging coronavirus infections. J Pathol. 2015 January; 235(2):185-95.
18. Gonzalvo R, Martf-Sistac O, Blanch L, López-Aguilar J. Bench-to-bedside review: brain-lung interaction in the critically ill—a pending issue revisited. Crit Care Lond Engl. 2007; 11(3):216.
19. Dizier S, Forel J-M, Ayzac L, Richard J-C, Hraiech S, Lehingue S, et al. Early Hepatic Dysfunction Is Associated with a Worse Outcome in Patients Presenting with Acute Respiratory Distress Syndrome: A Post-Hoc Analysis of the ACURASYS and PROSEVA Studies. PloS One. 2015; 10(12):e0144278.
20. Villacrés S M, Medar S S, Aydin S I. Acute Kidney Injury in Children With Acute Respiratory Failure. Clin Pediatr (Phila). 2018; 57(11):1340-8.
21. Atam V, Singh A S, Yathish B E, Das L. Acute pancreatitis and acute respiratory distress syndrome complicating Plasmodium vivax malaria. J Vector Borne Dis. 2013 June; 50(2):151-4.
22. Fox S E, Akmatbekov A, Harbert J L, Li G, Brown J Q, Heide R S V. Pulmonary and Cardiac Pathology in Covid-19: The First Autopsy Series from New Orleans. medRxiv. 2020 Apr. 10; 2020.04.06.20050575.
23. Ciceri F, Beretta L, Scandroglio A M, Colombo S, Landoni G, Ruggeri A, et al. Microvascular COVID-19 lung vessels obstructive thromboinflammatory syndrome (MicroCLOTS): an atypical acute respiratory distress syndrome working hypothesis. Crit Care Resusc J Australas Acad Crit Care Med. 2020 Apr. 15;
24. Tee A, Wong A, Yusuff T, Rao D, Sidhu P. Contrast-enhanced ultrasound (CEUS) of the lung reveals multiple areas of microthrombi in a COVID-19 patient. Intensive Care Med. 2020 May 18;
25. Jung E M, Stroszczynski C, Jung F. Contrast enhanced ultrasonography (CEUS) to detect abdominal microcirculatory disorders in severe cases of COVID-19 infection: First experience. Clin Hemorheol Microcirc. 2020 Apr. 23;
26. Hernández-Fernández F, Sandoval Valencia H, Barbella-Aponte R A, Collado-Jiménez R, Ayo-Martin Ó, Barrena C, et al. Cerebrovascular disease in patients with COVID-19: neuroimaging, histological and clinical description. Brain. 2020 Oct. 1; 143(10):3089-103.
27. Xie Y, Wang X, Yang P, Zhang S. COVID-19 Complicated by Acute Pulmonary Embolism. Radiol Cardiothorac Imaging. 2020 Apr. 1; 2(2):e200067.
28. Gao Y, Li T, Han M, Li X, Wu D, Xu Y, et al. Diagnostic utility of clinical laboratory data determinations for patients with the severe COVID-19. J Med Virol. 2020 Mar. 17;
29. Zhou F, Yu T, Du R, Fan G, Liu Y, Liu Z, et al. Clinical course and risk factors for mortality of adult inpatients with COVID-19 in Wuhan, China: a retrospective cohort study. Lancet Lond Engl. 2020 28; 395(10229):1054-62.
30. Tang N, Li D, Wang X, Sun Z. Abnormal coagulation parameters are associated with poor prognosis in patients with novel coronavirus pneumonia. J Thromb Haemost JTH. 2020; 18(4):844-7.
31. Danzi G B, Loffi M, Galeazzi G, Gherbesi E. Acute pulmonary embolism and COVID-19 pneumonia: a random association? Eur Heart J. 2020 Mar. 30;
32. Rotzinger D C, Beigelman-Aubry C, von Garnier C, Qanadli S D. Pulmonary embolism in patients with COVID-19: Time to change the paradigm of computed tomography. Thromb Res. 2020 Apr. 11; 190:58-9.
33. Panigada M, Bottino N, Tagliabue P, Grasselli G, Novembrino C, Chantarangkul V, et al. Hypercoagulability of COVID-19 patients in Intensive Care Unit. A Report of Thromboelastography Findings and other Parameters of Hemostasis. J Thromb Haemost JTH. 2020 Apr. 17;
34. Escher R, Breakey N, Lämmle B. Severe COVID-19 infection associated with endothelial activation. Thromb Res. 2020 Apr. 15; 190:62.
35. Lippi G, Plebani M, Henry B M. Thrombocytopenia is associated with severe coronavirus disease 2019 (COVID-19) infections: A meta-analysis. Clin Chim Acta Int J Clin Chem. 2020 Mar. 13; 506:145-8.
36. Yang M, Ng M H L, Li C K. Thrombocytopenia in patients with severe acute respiratory syndrome (review). Hematol Amst Neth. 2005 April; 10(2):101-5.
37. Yamamoto K, de Waard V, Fearns C, Loskutoff D J. Tissue distribution and regulation of murine von Willebrand factor gene expression in vivo. Blood. 1998 Oct. 15; 92(8):2791-801.
38. Arya M, Anvari B, Romo G M, Cruz M A, DongJ-F, McIntire L V, et al. Ultralarge multimers of von Willebrand factor form spontaneous high-strength bonds with the platelet glycoprotein Ib-IX complex: studies using optical tweezers. Blood. 2002 Jun. 1; 99(11):3971-7.
39. Ware L B, Conner E R, Matthay M A. von Willebrand factor antigen is an independent marker of poor outcome in patients with early acute lung injury. Crit Care Med. 2001 December; 29(12):2325-31.
40. Ware L B, Eisner M D, Thompson B T, Parsons P E, Matthay M A. Significance of von Willebrand factor in septic and nonseptic patients with acute lung injury. Am J Respir Crit Care Med. 2004 Oct. 1; 170(7):766-72.
41. Dong J, Moake J L, Nolasco L, Bernardo A, Arceneaux W, Shrimpton C N, et al. ADAMTS-13 rapidly cleaves newly secreted ultralarge von Willebrand factor multimers on the endothelial surface under flowing conditions. Blood. 2002 Dec. 1; 100(12):4033-9.
42. Padilla A, Moake J L, Bernardo A, Ball C, Wang Y, Arya M, et al. P-selectin anchors newly released ultralarge von Willebrand factor multimers to the endothelial cell surface. Blood. 2004 Mar. 15; 103(6):2150-6.
43. Huang J, Roth R, Heuser J E, Sadler J E. Integrin alpha(v)beta(3) on human endothelial cells binds von Willebrand factor strings under fluid shear stress. Blood. 2009 Feb. 12; 113(7):1589-97.
44. Tremblay D, van Gerwen M, Alsen M, Thibaud S, Kessler A J, Venugopal S, et al. Impact of anticoagulation prior to COVID-19 infection: a propensity score-matched cohort study. Blood. 2020 May 27;
45. Bernardo A, Ball C, Nolasco L, Moake J F, Dong J. Effects of inflammatory cytokines on the release and cleavage of the endothelial cell-derived ultralarge von Willebrand factor multimers under flow. Blood. 2004 Jul. 1; 104(1):100-6.
46. Westwood J-P, Langley K, Heelas E, Machin S J, Scully M. Complement and cytokine response in acute Thrombotic Thrombocytopenic Purpura. Br J Haematol. 2014 March; 164(6):858-66.
47. Goshua G, Pine A B, Meizlish M L, Chang C-H, Zhang H, Bahel P, et al. Endotheliopathy in COVID-19-associated coagulopathy: evidence from a single-centre, cross-sectional study. Lancet Haematol [Internet]. 2020 Jun. 30 [cited 2020 Jul. 8]; 0(0). Available from: https://www.thelancet.com/journals/lanhae/article/PIIS2352-3026(20)30216-7/abstract
48. Rauch A, Labreuche J, Lassalle F, Goutay J, Caplan M, Charbonnier L, et al. Coagulation biomarkers are independent predictors of increased oxygen requirements in COVID-19. J Thromb Haemost [Internet]. [cited 2020 Aug. 26]; n/a(n/a). Available from: http://onlinelibrary.wiley.com/doi/abs/10.1111/jth.15067
49. Ladikou E E, Sivaloganathan H, Milne K M, Arter W E, Ramasamy R, Saad R, et al. Von Willebrand factor (vWF): marker of endothelial damage and thrombotic risk in COVID-19? Clin Med Lond Engl. 2020 Jul. 21;
50. Helms J, Tacquard C, Severac F, Leonard-Lorant I, Ohana M, Delabranche X, et al. High risk of thrombosis in patients with severe SARS-CoV-2 infection: a multicenter prospective cohort study. Intensive Care Med. 2020; 406(6):1089-98.
51. Huisman A, Beun R, Sikma M, Westerink J, Kusadasi N. Involvement of ADAMTS13 and von Willebrand factor in thromboembolic events in patients infected with SARS-CoV-2. Int J Lab Hematol. 2020 October; 42(5):e211-2.
52. Mancini I, Baronciani L, Artoni A, Colpani P, Biganzoli M, Cozzi G, et al. The ADAMTS13-von Willebrand factor axis in COVID-19 patients. J Thromb Haemost JTH. 2020 Nov. 23;
53. Philippe A, Chocron R, Gendron N, Bory O, Beauvais A, Peron N, et al. Circulating Von Willebrand factor and high molecular weight multimers as markers of endothelial injury predict COVID-19 in-hospital mortality. Angiogenesis. 2021 Jan. 15; 1-13.
54. Goudot G, Chocron R, Augy J-L, Gendron N, Khider L, Debuc B, et al. Predictive Factor for COVID-19 Worsening: Insights for High-Sensitivity Troponin and D-Dimer and Correlation With Right Ventricular Afterload. Front Med. 2020; 7:586307.
55. Gattinoni L, Coppola S, Cressoni M, Busana M, Rossi S, Chiumello D. Covid-19 Does Not Lead to a “Typical” Acute Respiratory Distress Syndrome. Am J Respir Crit Care Med. 2020 Mar. 30;
56. Lippi G, Wong J, Henry B M. Hypertension and its severity or mortality in Coronavirus Disease 2019 (COVID-19): a pooled analysis. Pol Arch Intern Med. 2020 Mar. 31;
57. de Vries P S, van Herpt T T W, Ligthart S, Hofman A, Ikram M A, van Hoek M, et al. ADAMTS13 activity as a novel risk factor for incident type 2 diabetes mellitus: a population-based cohort study. Diabetologia. 2017; 60(2):280-6.
58. Sedaghat S, de Vries P S, Boender J, Sonneveld M A H, Hoorn E J, Hofman A, et al. von Willebrand Factor, ADAMTS13 Activity, and Decline in Kidney Function: A Population-Based Cohort Study. Am J Kidney Dis Off J Natl Kidney Found. 2016 November; 68(5):726-32.
59. Sonneveld M A H, Franco O H, Ikram M A, Hofman A, Kavousi M, de Maat M P M, et al. Von Willebrand Factor, ADAMTS13, and the Risk of Mortality: The Rotterdam Study. Arterioscler Thromb Vasc Biol. 2016; 36(12):2446-51.
60. Sonneveld M a. H, Kavousi M, Ikram M A, Hofman A, Rueda Ochoa O L, Turecek P L, et al. Low ADAMTS-13 activity and the risk of coronary heart disease—a prospective cohort study: the Rotterdam Study. J Thromb Haemost JTH. 2016; 14(11):2114-20.
61. Sonneveld M A H, de Maat M P M, Portegies M L P, Kavousi M, Hofman A, Turecek P L, et al. Low ADAMTS13 activity is associated with an increased risk of ischemic stroke. Blood. 2015 Dec. 17; 126(25):2739-46.
62. Bongers T N, de Bruijne E L E, Dippel D W J, de Jong A J, Deckers J W, Poldermans D, et al. Lower levels of ADAMTS13 are associated with cardiovascular disease in young patients. Atherosclerosis. 2009 November; 207(1):250-4.
63. Bongers T N, de Maat M P M, van Goor M-L P J, Bhagwanbali V, van Vliet HHDM, Gómez Garcia E B, et al. High von Willebrand factor levels increase the risk of first ischemic stroke: influence of ADAMTS13, inflammation, and genetic variability. Stroke. 2006 November; 37(11):2672-7.
64. Bongers T N, Emonts M, de Maat M P M, de Groot R, Lisman T, Hazelzet J A, et al. Reduced ADAMTS13 in children with severe meningococcal sepsis is associated with severity and outcome. Thromb Haemost. 2010 June; 103(6):1181-7.
65. Bhogal P, Jensen M, Collins G, Spooner O, Makalanda L, Hart D, et al. Letter in response to: Coagulation markers are independent predictors of increased oxygen requirements and thrombosis in COVID-19. J Thromb Haemost. 2020; 18(12):3382-4.
66. Sobel M, McNeill P M, Carlson P L, Kermode J C, Adelman B, Conroy R, et al. Heparin inhibition of von Willebrand factor-dependent platelet function in vitro and in vivo. J Clin Invest. 1991 May; 87(5):1787-93.
67. Sobel M, Soler D F, Kermode J C, Harris R B. Localization and characterization of a heparin binding domain peptide of human von Willebrand factor. J Biol Chem. 1992 May 5; 267(13):8857-62.
69. van Haren F M P, Page C, Laffey J G, Artigas A, Camprubi-Rimblas M, Nunes O, et al. Nebulised heparin as a treatment for COVID-19: scientific rationale and a call for randomised evidence. Crit Care Lond Engl. 2020 Jul. 22; 24(1):454.
70. Dixon B, Campbell D J, Santamaria J D. Elevated pulmonary dead space and coagulation abnormalities suggest lung microvascular thrombosis in patients undergoing cardiac surgery. Intensive Care Med. 2008 July; 34(7):1216-23.
71. Dixon B, Schultz M J, Smith R, Fink J B, Santamaria J D, Campbell D J. Nebulized heparin is associated with fewer days of mechanical ventilation in critically ill patients: a randomized controlled trial. Crit Care Lond Engl. 2010; 14(5):R180.
72. Dixon B, Smith R, Santamaria J D, Orford N R, Wakefield B J, Ives K, et al. A trial of nebulised heparin to limit lung injury following cardiac surgery. Anaesth Intensive Care. 2016 January; 44(1):28-33.
73. Dixon B, Schultz M J, Hofstra J J, Campbell D J, Santamaria J D. Nebulized heparin reduces levels of pulmonary coagulation activation in acute lung injury. Crit Care Lond Engl. 2010; 14(5):445.
74. Clausen T M, Sandoval D R, Spliid C B, Pihl J, Perrett H R, Painter C D, et al. SARS-CoV-2 Infection Depends on Cellular Heparan Sulfate and ACE2. Cell. 2020 Nov. 12; 183(4):1043-1057.e15.
75. Liu L, Chopra P, Li X, Bouwman K M, Tompkins S M, Wolfert M A, et al. Heparan sulfate proteoglycans as attachment factor for SARS-CoV-2. bioRxiv. 2021 Jan. 4; 2020.05.10.087288.

1. A medium molecular weight heparin for use in the treatment of endotheliopathy in a patient having a plasma von Willebrand factor antigen to ADAMTS13 ratio of at least about 2.

2. The medium molecular weight heparin according to claim 1, wherein the medium molecular weight heparin inhibits von Willebrand factor.

3. The medium molecular weight heparin according to claim 2, wherein the medium molecular weight heparin inhibits multimers of von Willebrand factor.

4. The medium molecular weight heparin according to claim 1, wherein the medium molecular weight heparin inhibits the binding of platelets to von Willebrand factor.

5. The medium molecular weight heparin according to claim 1, wherein the medium molecular weight heparin has a mass in the range of greater than about 8000 Da (g/mol) to about 13 000 Da (g/mol).

6. The medium molecular weight heparin according to claim 1, wherein the medium molecular weight heparin comprises at least three units of a IdoA2S-GlcNS6S disaccharide.

7. The medium molecular weight heparin according to claim 1, wherein the treatment of endotheliopathy inhibits the haematogenous spread of cancer.

8. The medium molecular weight heparin for the use in the treatment of a disease or condition in a patient, wherein the patient has an endotheliopathy characterized by a plasma von Willebrand factor antigen to ADAMTS13 ratio of at least about 2.

9. The medium molecular weight heparin according to claim 8, wherein the disease or condition is COVID-19, viral infection, acute respiratory distress syndrome, cancer, bacterial infection, septicaemia, cardiovascular disease, diabetes mellitus, trauma, burns, inhalational injury, drug reactions, haematological conditions, subarachnoid haemorrhage, or aneurysmal diseases.

10. The medium molecular weight heparin according to claim 9, wherein the endotheliopathy is caused by viral infection.

11. The medium molecular weight heparin according to claim 9, wherein the endotheliopathy is caused by cancer.

12. The medium molecular weight heparin according to claim 8, wherein the medium molecular weight heparin is administered by an administration method selected from the group consisting of: parenteral, subcutaneous, subcutaneous, depot form, for example depot injection, intravenous, intramuscular, intrathecal, intradermal, intraarterial, intraarticular, cutaneous, transcutaneous, intra-osseus, or inhalation.

13. The medium molecular weight heparin according to claim 12, wherein the administration method is subcutaneous.

14. The medium molecular weight heparin according to claim 12, wherein the administration method is intravenous.

15. The medium molecular weight heparin according to claim 12, wherein the administration method is intramuscular.

16. The medium molecular weight heparin according to claim 12, wherein the administration method is inhalation.

17. The medium molecular weight heparin according to claim 8, wherein the medium molecular weight heparin is administered at a dose of about 0.01 mg/kg to about 100 mg/kg.

18. The medium molecular weight heparin according to claim 12, wherein the medium molecular weight heparin is administered as a single dose or a continuous dose.

19. The medium molecular weight according to claim 1, wherein the medium molecular weight heparin is comprised in a pharmaceutical formulation.

20. The medium molecular weight according to claim 19, wherein the pharmaceutical formulation comprises an excipient.

21. The medium molecular weight according to claim 20, wherein the excipient is selected from the group consisting of solvents, co-solvents, buffers, stabilisers, antioxidants, preservatives, chelating agents, emulsifiers, flavourings, lubricants, suspending agents, tonicity adjusting agents, surfactants, solubilising agents, suspending aids, dispersion agents, humectants, thickeners, colouring agent, wetting agent, anti-foaming agent, viscosity modifier, sweeteners and combinations thereof.

22. The medium molecular weight heparin according to claim 19, wherein the pharmaceutical formulation comprises an additional active.

23. The medium molecular weight heparin-according to claim 1, wherein the medium molecular weight heparin comprises a chemical modification.

24. The medium molecular weight heparin according to claim 23, wherein the chemical modification comprises N-acetylation, N-deacetylation, N-sulfation, O-sulfation, 2-O desulfation, complete desulfation, or combinations thereof.

25. A kit comprising a medium molecular weight heparin according to claim 1.

26. A method of treating endotheliopathy, the method comprising administering to a patient in need of treatment a therapeutically effective amount of medium molecular weight heparin, wherein the patient has a plasma von Willebrand factor antigen to ADAMTS13 ratio of at least about 2.

27. The medium molecular weight heparin according to claim 2, wherein the medium molecular weight heparin fully inhibits von Willebrand factor-induced platelet aggregation at a concentration of 15 μM when measured by a ristocetin-induced platelet aggregation assay.

28. The medium molecular weight heparin according to claim 3, wherein the von Willebrand factor is ultra-large von Willebrand factor.

29. The medium molecular weight heparin according to claim 5, wherein the medium molecular weight heparin a mass of about 11 000 Da (g/mol).

30. The medium molecular weight heparin according to claim 10, wherein the viral infection is SARS-CoV-2.

31. The medium molecular weight heparin according to claim 11, wherein the cancer is leukaemia, lymphoma, myeloma, or a solid organ cancer.

32. The medium molecular weight heparin according to claim 17, wherein the medium molecular weight heparin is administered at a dose of about 0.01 mg/kg to about 10 mg/kg.

33. The medium molecular weight heparin according to claim 22, wherein the additional active agent comprises low molecular weight heparin.
